(19) United States
(12) Patent Application Publication
(10) Pub. No.: US 2006/0088882 A1
(43) Pub. Date: Apr. 27, 2006

(54) Methods for the treatment or prevention of obesity

(76) Inventors: Rakesh K Jain, Boston, MA (US); Dai Fukumura, Newton, MA (US)

Correspondence Address: CLARK & ELBING LLP, 101 FEDERAL STREET, BOSTON, MA, 02110, US

(21) Appl. No.: 10/519,248

(22) PCT Filed: Jun. 27, 2003
(86) PCT No.: PCT/US03/20591

§ 371 Date: Jan. 4, 2006

Related U.S. Application Data
(60) Provisional application No. 60/392,463, filed on Jun. 27, 2002.

(51) Int. Cl. C40B 40/10; A61K 39/395

(52) U.S. Cl. 435/7.1; 424/145.1

(57) ABSTRACT

The invention provides methods for the treatment or prevention of obesity. The invention also provides screening methods for determining whether a compound is useful for the treatment or prevention of obesity.

### BACKGROUND OF THE INVENTION

In general, the invention features methods for treating or preventing obesity by inhibiting vascular endothelial growth factor receptor 1 (VEGFR1, also called Flt-1) activity in a mammal (e.g., a human).

The prevalence of obesity is increasing world-wide. For example, the prevalence of obesity in the United States has increased 33 percent in the past decade alone (The Merck Manual of Medical Information, Berkow, ed., Pocket Books: New York, 1997). Additionally, people have the potential to acquire new fat cells from fat cell precursors throughout their life span. Unfortunately, many current therapies for obesity have limited long-term efficacy and may produce adverse side-effects. For example, people usually regain lost weight within three years. Thus, improved therapies are needed for obesity that have few adverse side-effects and greater efficacy.

### SUMMARY OF THE INVENTION

The purpose of the present invention is to provide improved therapies and methods for reducing or preventing undesired, excess body fat in a mammal. In particular, the invention features methods for identifying or selecting compounds that decrease VEGFR1 activity and thus are useful for decreasing the total body weight and percentage of body fat in a mammal (e.g., a human).

Accordingly, in a first aspect, the invention features a screening method for determining whether a compound is useful for treating, stabilizing, or preventing a higher than desired total body weight or a higher than desired percentage of body fat in a mammal. This method involves measuring VEGFR1 activity in a cell, tissue, or mammal in the presence and absence of the compound. The compound is determined to treat, stabilize, or prevent a higher than desired total body weight or a higher than desired percentage of body fat if the compound decreases VEGFR1 activity. In some embodiments, the method also includes administering the compound to a mammal in need of the treatment (e.g., an obese mammal or a mammal with excess fat). In certain embodiments, the compound is a member of a library of at least 5, 10, 15, 20, 30, 50, or more compounds, all of which are simultaneously administered to the cell, tissue, or mammal. Preferably, the compound decreases the level of VEGFR1 mRNA or protein, an activity of VEGFR1, the half-life of VEGFR1 mRNA or protein, the binding of VEGFR1 to a ligand or to another molecule (e.g., VEGF, VEGF-B, or PIGF), or the level or activity of a VEGFR1 ligand (e.g., VEGF, VEGF-B, or PIGF). In a preferred embodiment, the compound is a VEGFR1 receptor antagonist (e.g, an anti-VEGFR1 antibody). Preferably, the level of VEGFR1 mRNA or protein, an activity of VEGFR1 (e.g., kinase activity), the half-life of VEGFR1 mRNA or protein, the binding of VEGFR1 to a ligand or to another molecule (e.g., VEGF, VEGF-B or PIGF), or the level or activity of a VEGFR1 ligand (e.g., PIGF) decreases by at least 5, 10, 20, 30, 40, 50, 60, or 80%.

The invention also features improved methods for reducing or preventing undesired, excess body fat in a mammal. In particular, these methods involve administering a compound that decreases VEGFR1 activity to the mammal.

In one such aspect, the invention provides a method of treating, stabilizing, or preventing a higher than desired total body weight or a higher than desired percentage of body fat in a mammal (e.g., a human) that involves administering to the mammal a compound that decreases VEGFR1 activity in an amount sufficient to treat, reduce, or prevent a higher than desired total body weight or a higher than desired percentage of body fat. Preferably, the compound decreases the level of VEGFR1 mRNA or protein, an activity of VEGFR1, the half-life of VEGFR1 mRNA or protein, or the binding of VEGFR1 to a ligand or to another molecule (e.g., VEGF). In a preferred embodiment, the compound is a VEGFR1 receptor antagonist, such as an anti-VEGFR1 antibody. Preferred anti-VEGFR1 antibodies include MF-1 (ImClone) and anti-VEGFR1 antibodies that are substantially identical to a naturally-occurring antibody. Preferably, the compound inhibits angiogenesis and/or differentiation of preadipocytes into adipocytes in the mammal. In various embodiments, the compound, such as a quinazoline-urea, inhibits both VEGFR1 activity and VEGFR2 activity. Exemplary quinazoline-ureas include the compounds listed in FIGS. 11A-11C (Kubo et al., “Synthesis and Structure-Activity Relationship of Quinazoline-Urea Derivatives as Novel Orally Active VEGF Receptor Tyrosine Kinase Selective Inhibitors,” poster #913, AACR Meeting, 2002; Nakamura et al., “In Vitro Characterization of KRN633, a Novel Small Molecule Inhibitor of VEGF Receptor Tyrosine Kinases,” poster #876, AACR Meeting, 2002; Kamishohara et al., “A Novel Orally Active Dual Inhibitor of VEGF Receptor 1 and 2 Tyrosine Kinase KRN633: Anti-angiogenic and Anti-tumor Activity Against Human Solid Tumors,” poster #877, AACR Meeting, 2002).

In some embodiments, at least 2, 3, 4, 5, or more compounds that decrease VEGFR1 activity are administered to the mammal. Preferably, the one or more compounds are administered intravenously, parenterally, subcutaneously, intramuscularly, ophthalmicly, intraventricularly, intraperitoneally, orally, topically, or intranasally to the mammal. In preferred embodiments, the one or more compounds are administered using an extended release device. In other preferred embodiments, the methods according to the invention also include administering to the mammal an additional compound (e.g., a PPARγ inhibitor or antagonist such as a PPARγ ligand with an inhibitory effect) that inhibits angiogenesis, adipogenesis, or VEGF signaling. An exemplary PPARγ ligand that is a partial agonist of PPARγ transactivation is GW0072 from Glaxo Wellcome (Oberfield et al., Proc. Natl. Acad. Sci. USA 96:6102-6106, 1999). In particular embodiments, a compound (e.g., an antibody reactive with VEGF, VEGF-B, or PIGF) that decreases the level or activity of a VEGFR1 ligand (e.g., VEGF, VEGF-B, or PIGF) is also administered. In other embodiments, leptin or an inhibitor of leptin is not administered to the mammal.

Typically, the mammal treated with the methods of the invention is obese. Preferably, the percentage of body fat in the mammal decreases by at least 5, 10, 20, 30, 40, 50, 60, or 80%. In other preferred embodiments, total body weight of the mammal decreases by at least 5, 10, 20, 30, 40, 50, or 60%. Preferably, the number of cells other than adipocytes or endothelial cells decreases by less than 50, 40, 30, 20, 10 or 5%. In other preferred embodiments, the compound does not effect the viability or proliferation of cells other than adipocytes or endothelial cells.

With respect to the therapeutic methods of the invention, it is not intended that the administration of one or more compounds to a mammal be limited to a particular mode of administration, dosage, or frequency of dosing; the present invention contemplates all modes of administration, including intramuscular, intravenous, intraarticular, intralesional, subcutaneous, or any other route sufficient to provide a dose adequate to prevent or treat a higher than desired total body weight or a higher than desired percentage of body fat. These methods may be used to treat humans or any domesticated or farm animal. Examples of preferred mammals for use in the present methods include humans, cows, sheep, big-horn sheep, goats, buffalos, antelopes, oxen, horses, donkeys, mule, deer, elk, caribou, water buffalo, camels, llama, alpaca, rabbits, pigs, mice, rats, guinea pigs, hamsters, dogs, cats, and primates such as monkeys. The compound(s) may be administered to the mammal in a single dose or multiple doses. When multiple doses are administered, the doses may be separated from one another by, for example, one day, one week, one month, or one year. It is to be understood that, for any particular subject, specific dosage regimes should be adjusted over time according to the individual need and the professional judgment of the person administering or supervising the administration of the compositions. If desired, conventional treatments such as appetite suppressants, diet, and exercise may be used in combination with the compounds of the present invention.

As used herein by “treating, stabilizing, or preventing a higher than desired total body weight or a higher than desired percentage of body fat” is meant preventing or delaying an initial or subsequent occurrence of a higher than desired weight or percentage of body fat, or stabilizing or reducing a subject's total body weight or percentage of body fat. Obesity is typically classified as mild (i.e., 20 to 40% overweight based on the midpoint of the weight range for the subject's height on a standard height-weight table), moderate (i.e., 41 to 100% overweight), or severe (i.e., over 100% overweight). In some embodiments, the subject's body mass index (i.e., weight in kilograms divided by height in meters squared) is greater than 20, 25, 30, 35, 40, or 45 kg/m². In certain embodiments, the subject has an increased body weight or an increased percentage of body fat due to, at least in part, a hormonal or metabolic disorder (e.g., a thyroid disorder), brain damage (e.g., damage to the hypothalamus), an adverse side-effect from a medication, or a genetic factor. In some embodiments, the subject has a binge eating disorder, bulimia nervosa, or anorexia nervosa.

Desirably, administration of a compound of the present invention to the subject results in a decrease of at least 5, 10, 20, 30, 40, 50, or 60% in the subject's total body weight or weight due to body fat. Preferably, the decrease in muscle mass is less than 50, 40, 30, 20, 10, 5, or 3%. In other preferred embodiments, the decrease in body fat or total body weight leads to a decrease in blood pressure, incidence or severity of diabetes, or incidence or severity of coronary artery disease (e.g., heart attacks).

By “compound that decreases VEGFR1 activity” is meant a compound that decreases the level of VEGFR1 mRNA or protein, an activity of VEGFR1 (e.g., kinase activity), the half-life of VEGFR1 mRNA or protein, or the binding of VEGFR1 to a ligand or to another molecule (e.g., VEGF or PIGF), as measured using standard methods (see, for example, Ausubel et al., Current Protocols in Molecular Biology, Chapter 9, John Wiley & Sons, New York, 2000). VEGFR1 mRNA expression levels may be determined using standard RNase protection assays or in situ hybridization assays, and the level of VEGFR1 protein may be determined using standard Western or immunohistochemistry analysis with an anti-VEGFR1 antibody (see, for example, Ausubel et al., supra). In other preferred embodiments, a compound that decreases VEGFR 1 activity reduces or stabilizes the level of mRNA or protein, or the phosphorylation level of a signal transduction protein. The mRNA expression levels, protein expression levels, or phosphorylation levels of signal transduction proteins downstream of VEGFR1 receptor activation, such as PLCγ, RasGAP, Grb2, SHP-2, p38 MAP kinase, FAK, RAFTK, paxillin, may also be measured using standard assays. The level of VEGFR1 activity may be determined by measuring the level, duration, or delayed onset of angiogenesis, differentiation of preadipocytes into adipocytes, or change in total body weight or percentage of body fat using standard assays, such as those described herein. Compounds that may be tested for their ability to decrease VEGFR1 activity include, but are not limited to, synthetic organic molecules, naturally occurring organic molecules, nucleic acid molecules, VEGFR1 antisense nucleic acids, VEGFR1 double stranded RNA molecules, biosynthetic proteins or peptides, naturally occurring peptides or proteins, anti-VEGFR1 antibodies, or dominant negative VEGFR1 proteins. Preferably, the compound decreases VEGFR1 activity by at least 20, 40, 60, 80, or 90%. In another preferred embodiment, the level of VEGFR1 activity is at least 2, 3, 5, 10, 20, or 50-fold lower in the presence of the compound. In preferred embodiments, the decrease in VEGFR1 activity is at least 2, 5, 10, or 20-fold greater that the decrease in activity of another VEGFR receptor, such as VEGFR2.

By “decreasing expression or activity” is meant decreasing expression or activity, for example, of a protein or nucleic acid, relative to control conditions. The modulation in expression or activity is preferably a decrease of at least 20, 40, 50, 75, 90, 100, 200, 500, or even 1000%. In various embodiments, transcription, translation, mRNA or protein stability, or the binding of the mRNA or protein to other molecules in vivo is decreased by the therapy. The level of mRNA may be determined by standard Northern blot analysis, and the level of protein may be determined by standard Western blot analysis, such as the analyses described herein or those described by, for example, Ausubel et al. (Current Protocols in Molecular Biology, John Wiley & Sons, New York, 2000). In one embodiment, the level of a protein is determined by measuring the level of enzymatic activity, using standard methods. In another preferred embodiment, the level of mRNA, protein, or enzymatic activity is equal to or less than 20, 10, 5, or 2-fold above the corresponding basal level in from a control mammal with a normal percentage of body fat.

By “angiogenesis” is meant the formation of new blood vessels and/or the increase in the volume, diameter, length, or permeability of existing blood vessels, such as blood vessels in fat tissue or between fat tissue and surrounding tissue. Angiogenesis related diseases include, for example, diseases such as obesity that are associated with excessive blood vessel growth, an abnormal blood vessel network, and/or abnormal blood vessel remodeling during the initial and/or expanding phase of adipogenesis.

By “specifically binding a protein” is meant binding to the protein (e.g., VEGFR1), but not substantially binding to other molecules in a sample, e.g., a biological sample, that naturally includes the protein. Preferably, the amount antibody bound to VEGFR1 is at least 50%, 100%, 200%, 500%, or 1,000% greater than the amount of antibody bound to other proteins under the same conditions. In some embodiments, the amount of antibody bound to VEGFR1 is at least 2, 5, 10, or 20-fold more than the amount bound to another VEGF receptor, such as VEGFR2.

By “modified antibody” is meant an antibody having an altered amino acid sequence so that fewer antibodies and/or immune responses are elicited against the modified antibody when it is administered to a mammal such as a human. For example, the constant region of the antibody may be replaced with the constant region from a human antibody. For the use of the antibody in a mammal other than a human, an antibody may be converted to that species format.

By “bifunctional antibody” is meant an antibody that includes an antibody or a fragment of an antibody covalently linked to a different antibody or a different fragment of an antibody. In one preferred embodiment, both antibodies or fragments bind to different epitopes expressed on VEGFR1. Other preferred bifunctional antibodies bind to two different antigens, such as to both VEGFR1 and another protein involved in angiogenesis, VEGF signaling, or preadipocyte differentiation. Standard molecular biology techniques such as those described herein may be used to operably link two nucleic acids so that the fusion nucleic acid encodes a bifunctional antibody.

By “fragment” is meant a polypeptide having a region of consecutive amino acids that is identical to the corresponding region of an antibody of the invention but is less than the full-length sequence. The fragment has the ability to bind the same antigen as the corresponding antibody based on standard assays, such as those described herein. Preferably, the binding of the fragment to the antigen (e.g., VEGFR1) is at least 20, 40, 60, 80, or 90% of that of the corresponding antibody.

By “purified” is meant separated from other components that naturally accompany it. Typically, a factor is substantially pure when it is at least 50%, by weight, free from proteins, antibodies, and naturally-occurring organic molecules with which it is naturally associated. Preferably, the factor is at least 75%, more preferably, at least 90%, and most preferably, at least 99%, by weight, pure. A substantially pure factor may be obtained by chemical synthesis, separation of the factor from natural sources, or production of the factor in a recombinant host cell that does not naturally produce the factor. Proteins, vesicles, and organelles may be purified by one skilled in the art using standard techniques such as those described by Ausubel et al. (Current Protocols in Molecular Biology, John Wiley & Sons, New York, 2000). The factor is preferably at least 2, 5, or 10 times as pure as the starting material, as measured using polyacrylamide gel electrophoresis, column chromatography, optical density, HPLC analysis, or western analysis (Ausubel et al., supra). Preferred methods of purification include immunoprecipitation, column chromatography such as immunoaffinity chromatography, magnetic bead immunoaffinity purification, and panning with a plate-bound antibody.

By “substantially identical” is meant having a sequence that is at least 60, 70, 80, 90, or 100% identical to that of another sequence or to a naturally-occurring sequence. Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

By “mutation” is meant an alteration in a naturally-occurring or reference nucleic acid sequence, such as an insertion, deletion, frameshift mutation, silent mutation, nonsense mutation, or missense mutation. Preferably, the amino acid sequence encoded by the nucleic acid sequence has at least one amino acid alteration from a naturally-occurring sequence. Examples of recombinant DNA techniques for altering the genomic sequence of a cell, embryo, fetus, or mammal include inserting a DNA sequence from another organism (e.g., a human) into the genome, deleting one or more DNA sequences, and introducing one or more base mutations (e.g., site-directed or random mutations) into a target DNA sequence.

The present invention provides a number of advantages related to reducing or stabilizing the amount of body fat in a mammal. These methods are desirable because they may be used to obtain a significant, long-term reduction in body fat. The therapies of the invention are expected to have few, if any, adverse side-effects.

Other features and advantages of the invention will be apparent from the following detailed description and from the claims.

### BRIEF DESCRIPTION OF THE DRAWINGS

The application file contains drawings executed in color (FIGS. 1A, 1B, 13B, 13C, 13E, and 13F).

FIGS. 1A-1J illustrate angiogenesis and vessel remodeling during adipogenesis. FIG. 1A is a picture of a mouse dorsal skin chamber following 3T3-F442A cell implantation. FIG. 1B is a low power microscopic image of FIG. 1A. FIGS. 1C-1F are high power microscopic images of fluorescence contrast enhanced blood vessels at 7 days (FIG. 1C), 14 days (FIG. 1D), 21 days (FIG. 1E), and 28 days (FIG. 1F) after preadipocytes implantation. FIGS. 1G-1J are graphs of the quantitative analysis of blood vessels during adipogenesis: number of vessel segments in the high power view field (FIG. 1G); vascular length density (FIG. 1H); vessel diameter (FIG. 1I); and calculated blood vessel volume (FIG. 1J) (n=7).

FIGS. 2A-2H illustrate the effect of PPARγ inhibition on angiogenesis. FIGS. 2A-2D are fluorescence images of blood vessels at 21 days (FIGS. 2A and 2C), and 28 days (FIGS. 2 Band 2D) after mock- (FIGS. 2A and 2B) and PPARγ dominant negative- (FIGS. 2C and 2D) transfected preadipocytes implantation. FIGS. 2E-2H are graphs of the quantitative analysis of blood vessels: number of vessel segments in the high power view field (FIG. 2E); vascular length density (FIG. 2F); vessel diameter (FIG. 2G); and calculated blood vessel volume (FIG. 2H). There was no difference between two different control cells, mock- transfected preadipocytes (n=3) and EF1a-GFP 3T3-F442A cells (n=3). Thus, these two groups are combined as control for data presentation and statistical analysis. Filled circle, control (n=6); open square, PPARγ dominant negative-transfected preadipocytes (n=5). *P<0.01 as compared with corresponding control by two-tailed t-test.

FIGS. 3A-3H illustrate angiogenesis induced by 3T3-F442A preadipocyte cells (FIGS. 3A and 3B) and NIH 3T3 fibroblast cells (FIGS. 3C and 3D). FIGS. 3A-3D are pictures of a mouse dorsal skin chamber following cell implantation. Vessels were visualized at day 14 after implantation by fluorescence microscopy using FITC dextran (2M Dalton). FIGS. 3E-3H are graphs of quantitative analysis of blood vessels: number of vessel segments in the view field (FIG. 3E); vascular length density (FIG. 3F); vessel diameter (FIG. 3G); and calculated blood vessel volume (FIG. 3H). Closed diamonds denote 3T3-F442A preadipocytes (n=7); closed squares denote NIH 3T3 fibroblasts (n=4).

FIG. 4 is a vessel diameter histogram during adipogenesis. Each segment in FIG. 3G was categorized to a group depending on its diameter and shown as cumulative frequency distribution. The segments distributed extensively over a wide range at day 7. The distribution shifted leftward and the range became narrow as a result of vessel remodeling with continued adipogenesis. At day 28, most segments (92%) were distributed from 3 to 9 μm in diameter.

FIGS. 5A-5C are pictures of preadipocyte differentiation. FIG. 5A is a transillumination image of differentiated adipocytes in vitro. FIG. 5B is a fluorescence image of differentiated adipocytes in vivo. FIG. 5C is a picture showing the effect of PPARγ inhibition on adipogenesis. Preadipocytes (1×10⁵ cells) were plated in a 6-well plate and transfected at a multiplicity of infection of 10⁴ plaque forming units/cell using mock- (upper panels) or PPARγ-DN adenovirus (lower panels) and differentiation was promoted by using media containing 10% FBS. Oil Red O (0.3%, 1 hour at room temperature, Sigma., St. Louis, Mo.) staining was performed at day 12 after transfection (magnification, ×20), confirming the inhibition of adipogenesis by the PPARγ-DN construct in murine preadipocytes.

FIG. 6 is a picture of VEGF mRNA expression during adipocyte differentiation. Differentiation was initiated when the cell became confluent by addition of differentiation media. For control, the cells were cultured in the maintenance media without insulin. At day 8 and 12 after replacement of the media, total cellular RNA was obtained from 3T3-F442A adipocytes, and 10 μg aliquots were electrophoresed, blotted, and hybridized to the VEGF cDNA, 18S and 28S as described herein.

FIG. 7A and 7B illustrate the effect of VEGF on preadipocyte differentiation and proliferation. To investigate the effects of VEGF on the in vitro differentiation of preadipocytes, 3T3-F442A cells were grown to confluence in media supplemented with calf serum (FCS, maintenance media), and exposed to increasing concentrations of murine recombinant VEGF₁₆₄ (R&D Systems, Minneapolis, Minn.) from 0-100 ng/ml. Mouse recombinant VEGF₁₆₄ did not induce differentiation in preadipocytes cultured in 10% FCS (maintenance media), and did not increase the differentiation rate in cells treated with 10% FBS (differentiation media) (FIG. 7A). Adipocyte differentiation was assessed by Oil Red O staining.

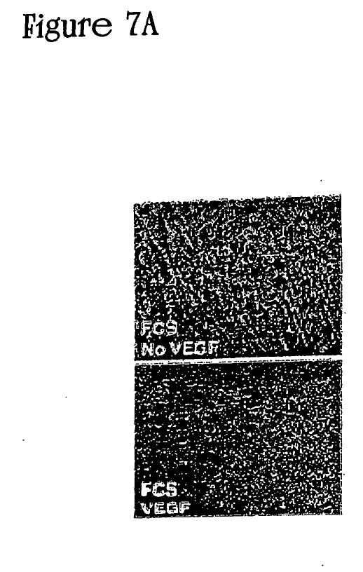

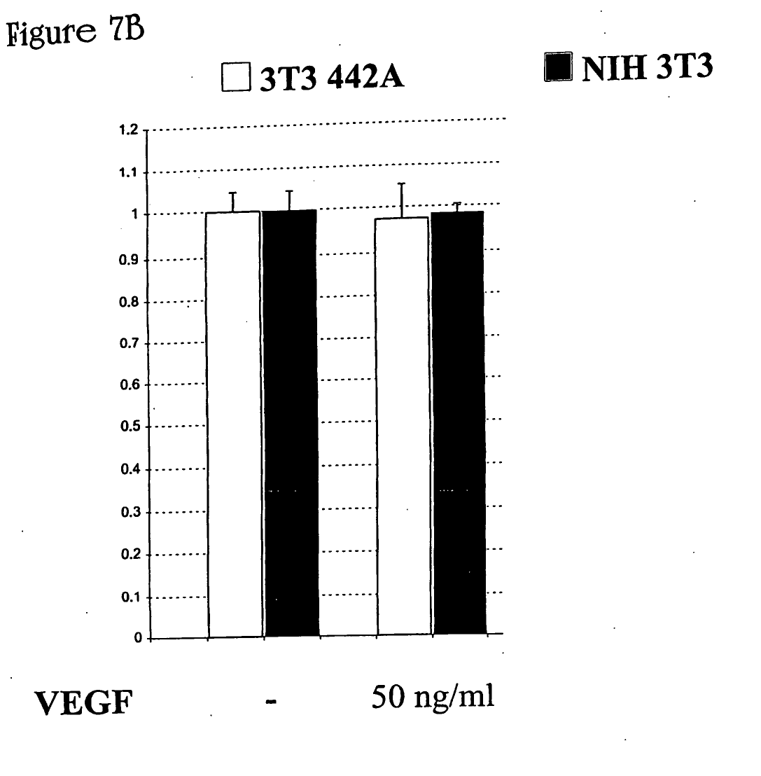

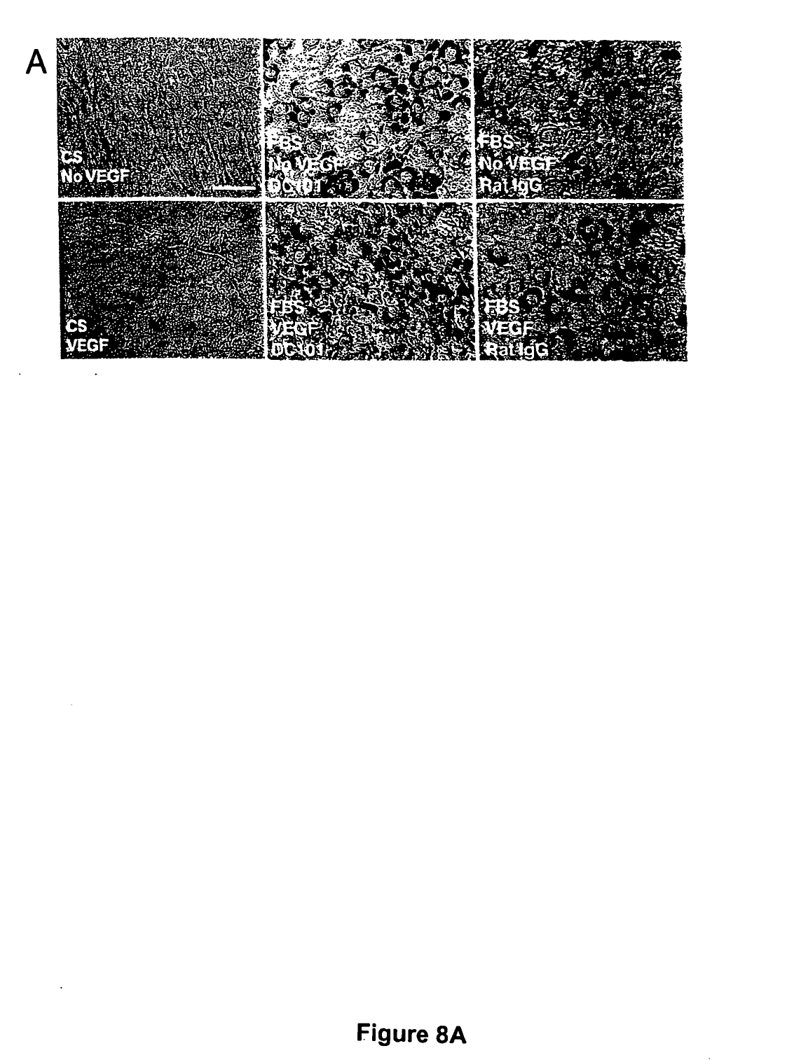

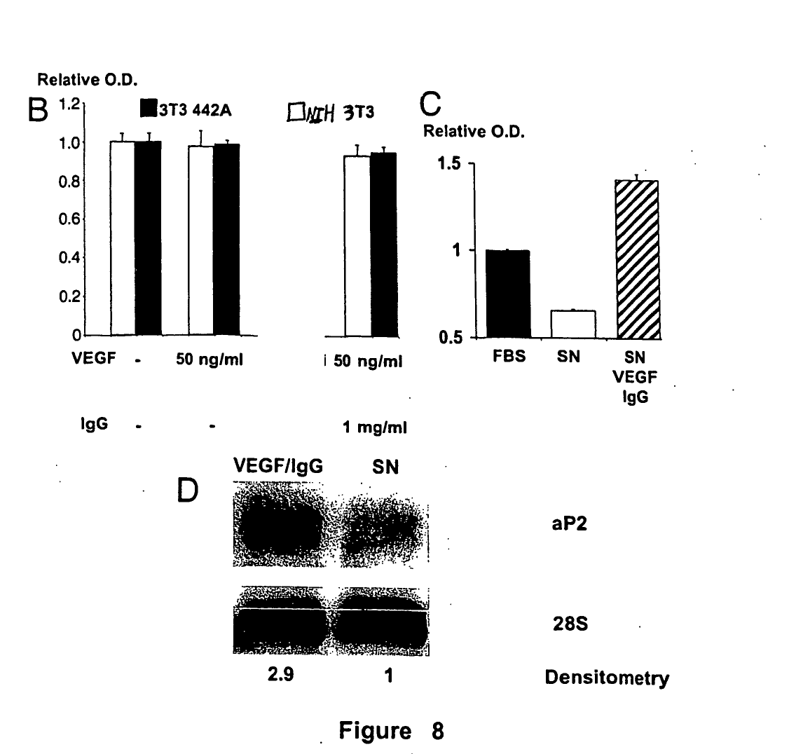

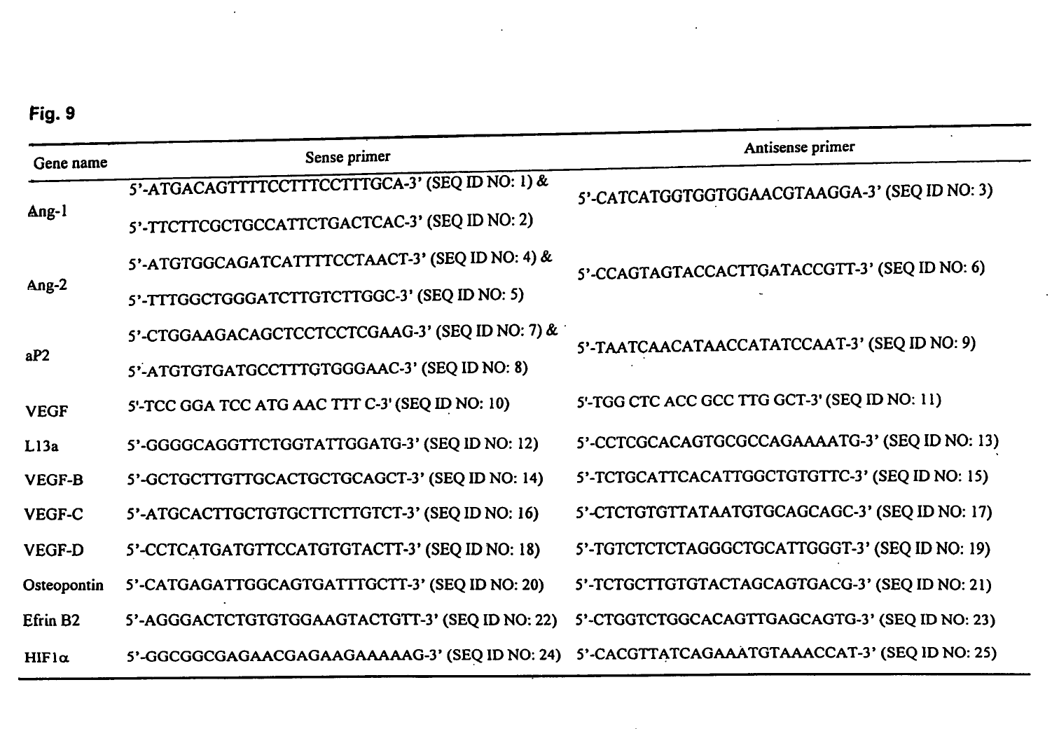

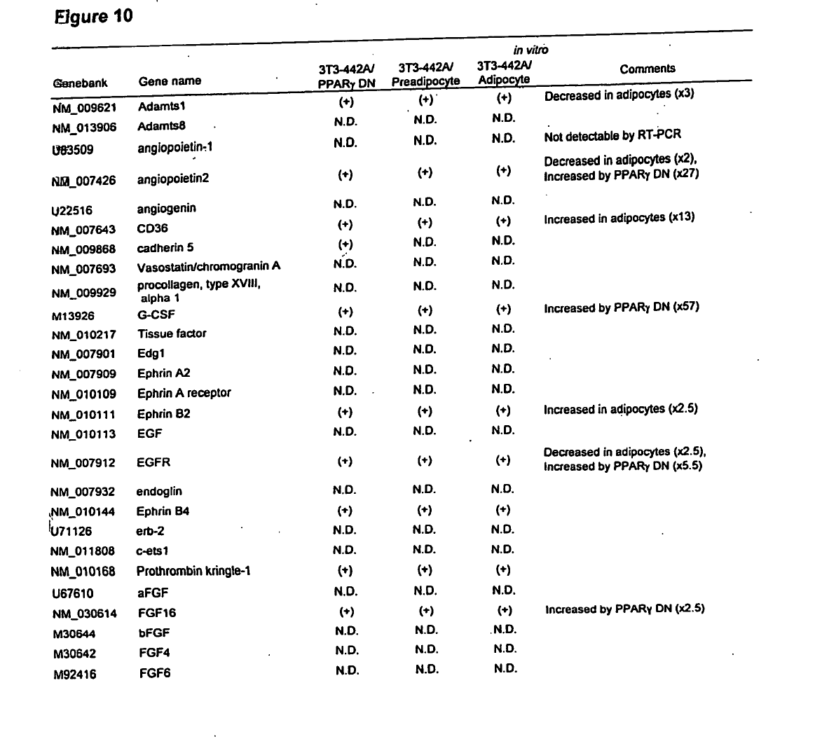

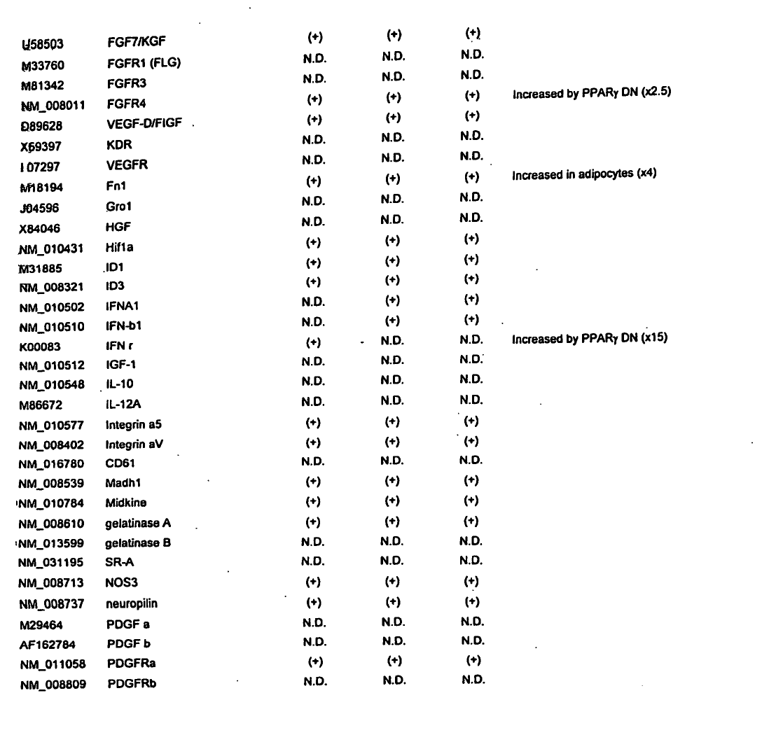

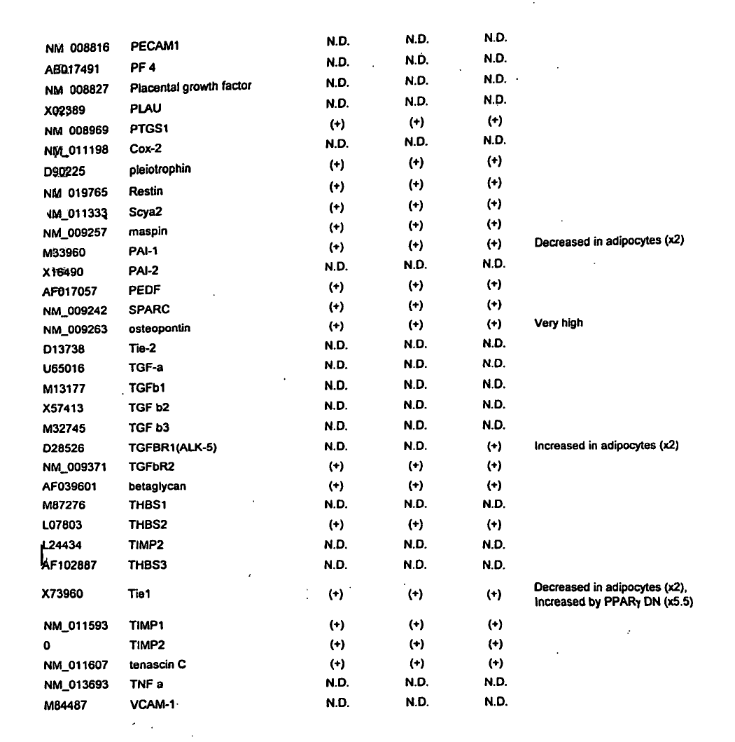

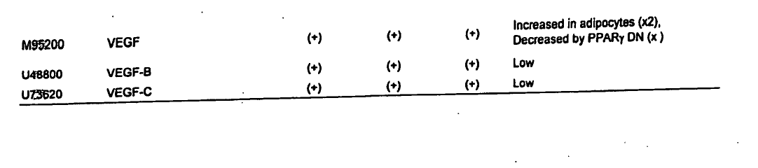

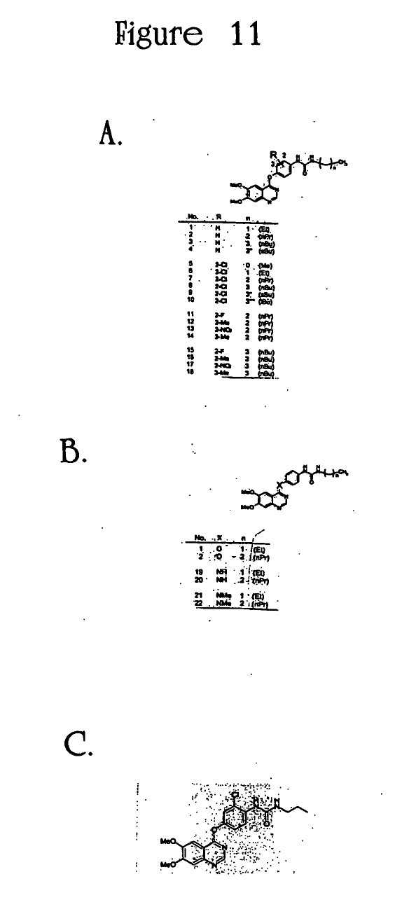

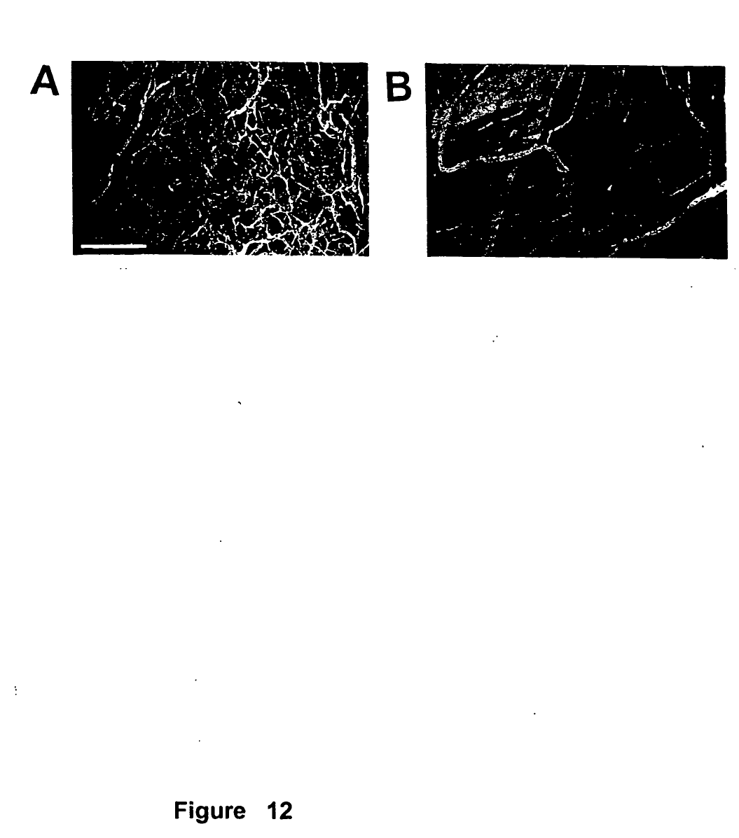

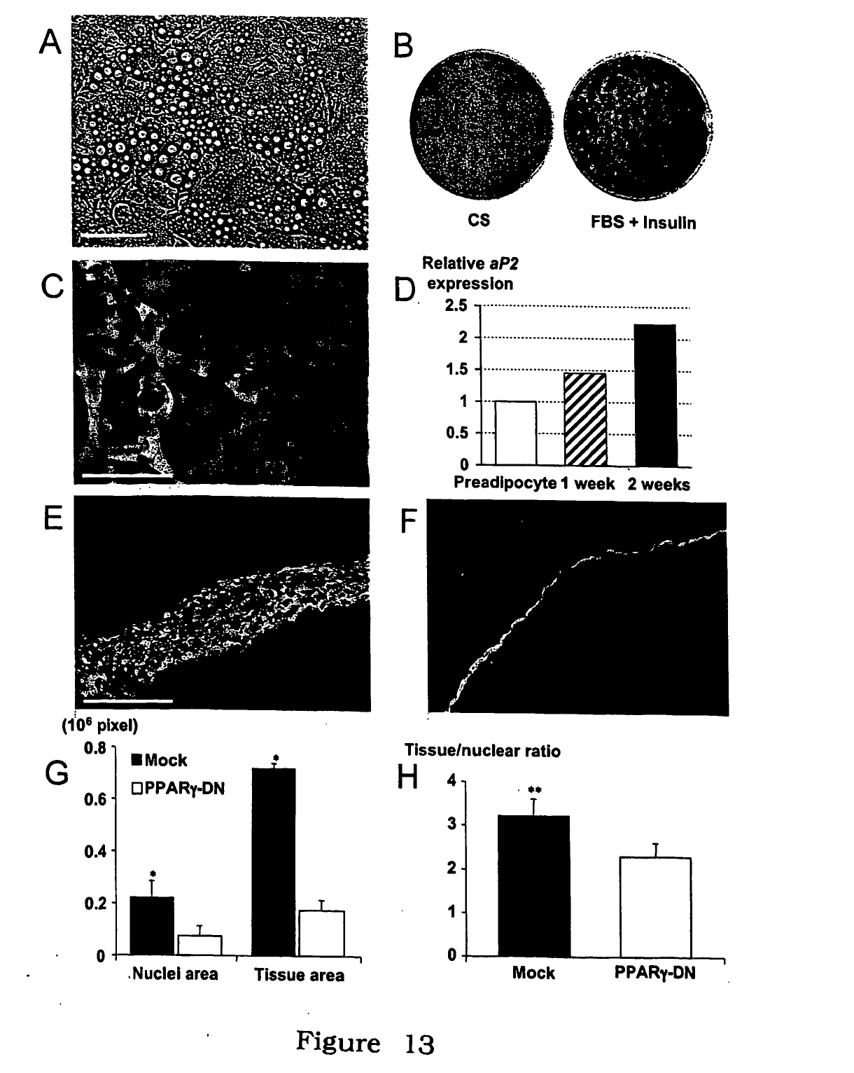

For proliferation assays, 500 preadipocytes and fibroblasts were plated in 96 well plates, and mouse recombinant VEGF₁₆₄ (50 ng/ml) and PBS were added (FIG. 7B). An MTT assay was performed at day 4, when the cells were still subconfluent in all wells. Culture media were changed with 100 μl of fresh media, and 10 μl of sterile tetrazolium salt, MTT (3[4,5-dimethylthiazol-2-yl]-2,5-diphenyl-tetrazolium bromide, Sigma) was added in each well and incubated for four hours at 37° C. Finally, 100 μl of 10% SDS were added, and after incubation at 37° C. overnight, the plate was read at 490 nm. The optical density values were normalized to that of the PBS treated cells and used as a measure of viability.

FIGS. 8A-8D illustrate the role of VEGF signaling in preadipocyte differentiation and proliferation. Confluent 3T3-F442A cells were cultured for II days with either 10% calf serum (CS, maintenance media) with or without VEGF, or 10% FBS (differentiation media) with or without VEGF (50 ng/ml). Rat IgG (1 μg/ml) was also added (FIG. 8A). Then, cells were stained with Oil Red-O. Mouse recombinant VEGF did not induce differentiation in preadipocytes cultured in 10% CS (maintenance media), and did not increase the differentiation rate in cells treated with 10% FBS (differentiation media). Addition of IgG to the culture media had no effect on in vitro adipogenesis. The bar indicates 50 μm. For MTT assays, mouse recombinant VEGF (50 ng/ml) and PBS, and rat IgG (1 μg/ml) were used. MTT assay was performed at day 4. The optical density values were normalized to that of the PBS treated cells and were used as a measure of viability (FIGS. 8B and 8C). MTT assay for preadipocytes after four days of culture with endothelial conditioned media. “FBS” denotes non-conditioned culture media with 10% FBS; “SN” denotes supernatant, endothelial conditioned media cultured with 10% FBS but no other additives; “SN/VEGF/IgG” denotes endothelial conditioned media cultured with 10% FBS, VEGF (50 ng/ml) and non-specific IgG (5 μg/ml). Data are normalized to the control condition (FBS). Error bars represent standard error. In vitro gene expression of aP2 in preadipocytes was measured after 11 days of culture with endothelial conditioned media (FIG. 8D). “VEGF/IgG” denotes endothelial conditioned media cultured with 10% FBS, VEGF (50 ng/ml), and non-specific IgG (dose); “SN” denotes endothelial conditioned media with 10% FBS and no other additives. The fold-increase in aP2 expression by differentiating preadipocytes was calculated by densitometry, normalizing to the control condition (SN).

FIG. 9 is a table of PCR primers (SEQ ID NOs: 1-25). Northern blots were probed with PCR-generated cDNA fragments. Nested primers were used to generate specific amplification products. Primers for PCR were synthesized based on Ang1, Ang2, and aP2 mouse sequences (GenBank accession numbers AAB50558, NM_{—}007426, and NM_{—}024406). Primers for RT-PCR were synthesized based on the GenBank sequence information. These primers were designed to amplify fragments of about 300 basepairs. Twenty five cycles each of 20 seconds at 93° C., 20 seconds at 55° C., and 30 seconds at 72°0 C. were performed. PCR products were resolved by 2% agarose gel. The gel was stained with ethidium bromide, and bands were visualized on an UV transilluminator.

FIG. 10 is a table of angiogenic gene array analysis performed using Mouse Angiogenesis GEArray Q Series (Superarray Inc., Bethesda, Md.), which contains 96 genes known to be involved in angiogenesis, according to the manufacturer's instructions. “(+)” denotes detectable; in this case, intensity was higher than background, and hybridization was confirmed by visual inspection. “N.D.” denotes not detectable; in this case, intensity was lower or close to background. Samples are from cultured 3T3-442A cells. Preadipocytes were cultured in maintenance media (10% FCS). “PPAR-DN” denotes PPARγ dominant negative mutant receptor transduced cells cultured in maintenance media. Adipocytes were cultured in differentiation media (10% FBS). More than 2-fold difference in normalized intensity compared to preadipocytes is noted.

FIGS. 11A-11C are chemical structures of quinazoline-ureas that can be used in the methods of the invention (Kubo et al., supra; Nakamura et al., supra; Kamishohara et al., supra). FIG. 11C is the chemical structure of KRN633, which inhibits both VEGFR1 and VEGFR2. Asterisks denote branched alkyl groups.

FIGS. 12A and 12B illustrate angiogenesis and vessel remodeling during adipogenesis. Multiphoton laser-scanning microscopy images taken 28 days after 3T3-F442A preadipocyte implantation into a mouse dorsal skin chamber are shown. These pictures include the maximum intensity projection images of 31 optical slices with 5 μm increments. FIG. 12A shows the top 150 μm de novo adipose tissue layer, and FIG. 12B shows the bottom 150 μm host subcutaneous layer. The bars indicate 200 μm.

FIGS. 13A-13H illustrate the effect of PPARγ-DN on differentiation of 3T3-F442A preadipocytes and de novo adipose tissue formation. FIG. 13A is a microscopic image of differentiated adipocytes in vitro (transillumination image), and FIG. 13B is macroscopic images of Oil Red O staining. FIG. 13C is a fluorescence image of differentiated adipocytes in vivo. To identify implanted cells in vivo, GFP labeled preadipocytes were used. Fat accumulated in GFP positive cells during conversion of the fibroblast-like shaped preadipocyte to a round-shaped adipocyte. This observation was facilitated by the granular fluorescence due to the fat droplet in the cytosol that was observed by multiphoton microscope (rhodamine dextran vessel enhancement). FIG. 13D is a graph of increasing aP2 levels, which indicate active adipogenesis upon s.c. cell implant. The values are based on quantitative densitometry of a Northern blot. Fluorescence microscopy in tissue sections from mock- (FIG. 13E) and PPARγ dominant negative- (FIG. 13F) transfected preadipocytes generated tissue was performed. Histological analyses of tissue expansion and cell size indicated an increased number of cells and tissue area in mock- preadipocytes tissue compared to PPARγ dominant negative- tissue (FIG. 13G). In contrast, the number of cells per tissue area is decreased in mock- preadipocyte generated tissue, documenting the increase in size of the cells in these tissues (FIG. 13H). * P<0.01 as compared with corresponding control by two-tailed t-test. **P<0.05 using the Wilcoxon rank-sum test. The bars indicate 50 μm (FIG. 13A), 50 μm (FIG. 13C), and 0.5 mm (FIGS. 13E and 13F), respectively.

### DETAILED DESCRIPTION

With an increasing incidence of obesity world-wide, more effective strategies are needed to control adipogenesis. We discovered that compounds that decrease VEGFR1 activity (e.g., anti-VEGFR1 antibodies) prevent or reduce excess or undesired body fat. Thus, a variety of compounds, such as anti-VEGFR1 antibodies or small molecules (e.g., organic molecules with a molecular weight less than 500, 400, 300, or 200 daltons), can be used to inhibit VEGFR1 activity for the treatment or prevention of obesity as described herein. Preferred VEGFR1 antagonists inhibit not only angiogenesis and vessel remodeling, but also preadipocyte differentiation.

Because the growth of any tissue requires the formation of new blood vessels, we characterized the link between preadipocyte differentiation and angiogenesis. To this end, we developed a novel model to visualize angiogenesis using intravital microscopy during adipose tissue formation from murine preadipocytes implanted in a mouse dorsal skin-fold chamber. Preadipocytes induced vigorous angiogenesis in vivo. The newly formed vessels subsequently remodeled into a mature network consisting of arterioles, capillaries, and venules. The differentiation of preadipocytes into adipocytes was confirmed by expression of the adipocyte-specific gene aP2. Inhibition of preadipocyte differentiation by blocking PPARγ signaling (e.g., by transfection of a PPARγ dominant negative into preadipocytes) retarded not only adipogenesis but also angiogenesis. VEGF signaling in the endothelial cells but not preadipocytes appears to mediate preadipocyte differentiation into adipocytes. These findings reveal a reciprocal regulation of adipogenesis and angiogenesis and suggest that blockade of VEGF signaling can inhibit in vivo adipose tissue formation. This animal model of angiogenesis and preadipocyte differentiation can be used to screen candidate compounds (e.g., known inhibitors of VEGFR1 activity, anti-VEGFR1 antibodies, or compounds of unknown function) for the ability to treat or prevent obesity as described herein.

These methods are described further below.

Production of Anti-VEGFR1 Antibodies for Treating or Preventing Obesity

Anti-VEGFR1 antibodies represent a preferred VEGFR-1 antagonist for use in the present invention. Anti-VEGFR1 antibodies can be generated using standard methods such as those described herein. If desired, the ability of an anti-VEGFR1 antibody to inhibit VEGFR1 activity can be confirmed before the antibody is administered to mammals (e.g., humans) for the treatment or prevention of obesity. For example, standard methods can be used to determine the ability of anti-VEGFR1 antibodies to decrease the level of VEGFR1 protein, an activity of VEGFR1 (e.g., kinase activity), the half-life of VEGFR1 protein, or the binding of VEGFR1 to a ligand or to another molecule (e.g., VEGF or PIGF). Anti-VEGFR1 antibodies can also be tested in animal or primate models, such as those described herein, to measure their effect on angiogenesis, adipogenesis, or total body fat in vivo.

For the preparation of polyclonal antibodies reactive with VEGFR1 for the treatment or prevention of obesity, one or more VEGFR1 proteins, fragments of VEGFR1 (e.g., an extra cellular domain of VEGFR1), or fusion proteins containing defined portions of VEGFR1 can be purified from natural sources (e.g., cultures of cells expressing VEGPR1) or synthesized in, e.g., mammalian, insect, or bacterial cells by expression of corresponding DNA sequences contained in a suitable cloning vehicle. Fusion proteins are commonly used as a source of antigen for producing antibodies. The antigenic proteins can be optionally purified, and then coupled to a carrier protein, mixed with Freund's adjuvant to enhance stimulation of the antigenic response in an inoculated animal, and injected into rabbits, mice, or other laboratory animals. Primary immunizations are carried out with Freund's complete adjuvant and subsequent immunizations performed with Freund's incomplete adjuvant. Following booster injections at bi-weekly intervals, the inoculated animals are then bled and the sera isolated. The sera is used directly or is purified prior to use by various methods, including affinity chromatography employing reagents such as Protein A-Sepharose, antigen-Sepharose, and anti-horse-Ig-Sepharose. Antibody titers can be monitored by Western blot and immunoprecipitation analyses using VEGFR1. Immune sera can be affinity purified using VEGFR1 coupled to beads. Antiserum specificity can be determined using a panel of proteins, such as VEGFR1 and other VEGF receptors.

Alternatively, monoclonal antibodies are produced by removing the spleen from the inoculated animal, homogenizing the spleen tissue, and suspending the spleen cells suspended in phosphate buffered saline (PBS). The spleen cells serve as a source of lymphocytes, some of which produce antibody of the appropriate specificity. These cells are then fused with permanently growing myeloma partner cells, and the products of the fusion plated into a number of tissue culture wells in the presence of selective agents, such as hypoxanthine, aminopterine, and thymidine (Mocikat, J. Immunol. Methods 225:185-189, 1999; Jonak et al., Hum. Antibodies Hybridomas 3:177-185, 1992; Srikumaran et al., Science 220:522, 1983). The wells can then be screened by ELISA to identify those containing cells making antibody capable of binding to VEGFR1, fragments, or mutants thereof. These cells can then be re-plated and, after a period of growth, the wells containing these cells can be screened again to identify antibody-producing cells. Several cloning procedures can be carried out until over 90% of the wells contain single clones that are positive for specific antibody production. From this procedure, a stable line of clones that produce the antibody are established. The monoclonal antibody can then be purified by affinity chromatography using Protein A Sepharose and ion-exchange chromatography, as well as variations and combinations of these techniques. Once produced, monoclonal antibodies are also tested for specific VEGFR1 recognition by ELISA, Western blot, and/or immunoprecipitation analysis (see, e.g., Kohler et al., Nature 256:495, 1975; Kohler et al., European Journal of Immunology 6:511, 1976; Kohler et al., European Journal of Immunology 6:292, 1976; Hammerling et al., In Monoclonal Antibodies and T Cell Hybridomas, Elsevier, New York, N.Y., 1981; Ausubel et al., supra).

As an alternate or adjunct immunogen to VEGFR1, peptides corresponding to relatively unique hydrophilic regions of VEGFR1 can be generated and coupled to keyhole limpet hemocyanin (KLH) through an introduced C-terminal lysine. Antiserum to each of these peptides can be similarly affinity-purified on peptides conjugated to BSA, and specificity tested by ELISA and Western blotting using peptide conjugates, and by Western blotting and immunoprecipitation using VEGFR1.

Antibodies of the invention are preferably produced using VEGFR1 amino acid sequences that do not reside within highly conserved regions, and that appear likely to be antigenic, as evaluated by criteria such as those provided by the Peptide Structure Program (Genetics Computer Group Sequence Analysis Package, Program Manual for the GCG Package, Version 7, 1991) using the algorithm of Jameson et al., CABIOS 4:181, 1988. These fragments can be generated by standard techniques, e.g., by PCR, and cloned into any appropriate expression vector. For example, GST fusion proteins can be expressed in E. coli and purified using a glutathione-agarose affinity matrix (Ausubel et al., supra). To minimize the potential for obtaining antisera that is non-specific or exhibits low-affinity binding to VEGFR1, two or three fusions may be generated for each fragment injected into a separate animal. Antisera are raised by injections in series, preferably including at least three booster injections.

In addition to intact monoclonal and polyclonal anti-VEGR1 antibodies, various genetically engineered antibodies and antibody fragments (e.g., F(ab′)2, Fab′, Fab, Fv, and sFv fragments) can be produced using standard methods. Truncated versions of monoclonal antibodies, for example, can be produced by recombinant methods in which plasmids are generated that express the desired monoclonal antibody fragment(s) in a suitable host.

Ladner (U.S. Pat. Nos. 4,946,778 and 4,704,692) describes methods for preparing single polypeptide chain antibodies. Ward et al., Nature 341:544-546, 1989, describes the preparation of heavy chain variable domain which have high antigen-binding affinities. McCafferty et al. (Nature 348:552-554, 1990) show that complete antibody V domains can be displayed on the surface of fd bacteriophage, that the phage bind specifically to antigen, and that rare phage (one in a million) can be isolated after affinity chromatography. Boss et al. (U.S. Pat. No. 4,816,397) describes various methods for producing immunoglobulins, and immunologically functional fragments thereof, that include at least the variable domains of the heavy and light chains in a single host cell. Cabilly et al. (U.S. Pat. No. 4,816,567) describes methods for preparing chimeric antibodies. In addition, the antibodies can be coupled to compounds, such as toxins or radiolabels.

Exemplary anti-VEGFR1 antibodies are described by Rockwell et al. (U.S. Pat. Nos. 5,874,542 and 6,365,157). Other anti-VEGFR1 antibodies can be readily produced using the methods described herein or the methods described by Rockwell.

Other Compounds for Treating or Preventing Obesity

As an alternative to anti-VEGFR1 antibodies, a variety of other compounds that inhibit VEGFR1 activity (e.g., synthetic organic molecules, naturally occurring organic molecules, nucleic acid molecules, VEGFR1 antisense nucleic acids, VEGFR1 double stranded RNA molecules, biosynthetic proteins or peptides, naturally occurring peptides or proteins, or dominant negative VEGFR1 proteins) can be used in the present invention. For example, compounds for the treatment or prevention of a higher than desired total body weight or a higher than desired percentage of body fat may be identified from large libraries of both natural product or synthetic (or semi-synthetic) extracts or chemical libraries according to methods known in the art. Compounds of unknown or known function can be tested for their ability to inhibit VEGFR1 activity. For example, known compounds that are currently used to treat other conditions can be assayed to determine whether they decrease VEGFR1 activity and thus are also useful for the treatment or prevention of obesity. Those skilled in the field or drug discovery and development will understand that the precise source of test extracts or compounds is not critical to the methods of the invention.

Accordingly, virtually any number of chemical extracts or compounds can be screened for their effect on reducing total body weight or body fat. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

When a crude extract is found to inhibit angiogenesis and/or adipogenesis, further fractionation of the positive lead extract is necessary to isolate chemical constituent responsible for the observed effect. Thus, the goal of the extraction, fractionation, and purification process is the careful characterization and identification of a chemical entity within the crude extract. Methods of fractionation and purification of such heterogeneous extracts are known in the art. If desired, compounds shown to be useful agents for the treatment or prevention of a higher than desired total body weight or a higher than desired percentage of body fat are chemically modified according to methods known in the art. Compounds identified as being of therapeutic value are subsequently analyzed using any standard animal model of angiogenesis, adipogenesis, or obesity known in the art.

Mouse Model of Angiogenesis and Adipogenesis for Identification of Compounds Useful to Treat or Prevent Obesity

The following mouse model of angiogenesis and adipogenesis was generated to further characterize the relationship between these processes and to provide an animal model that can be used to test the ability of compounds to treat or prevent obesity. Preferred compounds inhibit angiogenesis and/or adipogenesis in this mouse model.

In particular, 3T3-F442A murine preadipocytes were implanted into a mouse dorsal skin chamber, which is an in vivo transparent window model. This model enabled us to monitor in parallel the kinetics of angiogenesis and adipogenesis by intravital microscopy and gene expression patterns during fat tissue formation. Intravital microscopy is a powerful optical imaging technique that can be used to non-invasively monitor and quantify dynamic biological events including angiogenesis in real time with appropriate optical probes.

Generation of Mouse Model

To generate a mouse model of angiogenesis and adipogenesis, an established preadipocyte cell line (3T3-F442A), which is able to differentiate into mature adipocytes in culture, was used (Gregoire et al., Physiol. Rev. 78:783-809, 1998). 3T3-F442A cells also give rise to vascularized fat pads in immunodeficient mice (Mandrup et al., Proc. Natl. Acad. Sci. U.S.A. 94:4300-4305, 1997). 3T3-F442A preadipocytes (a generous gift from Dr. Bruce Spiegelman, Dana-Farber Cancer Institute, Boston, Mass.) and their parental cell line (NIH 3T3 fibroblasts) were maintained in Dulbecco's Minimum Essential Medium (DMEM, Gibco BRL, Grand Islands, N.Y.), supplemented with 10% calf serum, glucose, L-glutamine, penicillin, and streptomycin. For cell identification in vivo, preadipocytes were transfected by the calcium phosphate method with GFP under the EF1a promoter; these cells are referred to as GFP/3T3-F442A.

These preadipocytes were implanted in the dorsal skin-fold chamber (Jain et al., Nat. Rev. Cancer 2:266-276, 2002 and Leunig et al., Cancer Res. 52:6553-6560, 1992) of male, 8-12-weeks-old severe combined immunodeficient (SCID) mice to monitor angiogenesis during fat formation. In particular, dense cell pellets containing 2×10⁵ mouse preadipocytes (or NIH 3T3 fibroblasts as a control) were implanted in the center of the dorsal skinfold chamber. These mice were bred and maintained in a defined flora facility. In vivo microscopy using epifluorescence and multiphoton techniques was performed 1-2 times a week up to four weeks after the implantation followed by off-line analysis for vascular parameters as described previously (Jain et al., supra). Five random locations were observed for each animal and time point. Number of non-branching blood vessel segments (number of segments per unit area), functional vascular density (total length of perfused blood vessel per unit area), vessel diameter, vessel volume density (total of calculated blood vessel volume based on length and diameter of each segment per unit area) were determined as described elsewhere (Jain et al., supra). Angiogenesis and subsequent vessel remodeling were analyzed following cell implants of NIH 3T3, 3T3-F442A, GFP/3T3-F442A, GFP/3T3-F442A infected with a recombinant adenovirus encoding a PPARγ dominant negative (PPARγ DN) mutant receptor or mock adenovirus (Gurnell et al., J. Biol. Chem. 275:5754-5759, 2000), or GFP/3T3-F442A. Separate sets of windows were visualized using multiphoton laser-scanning microscopy as previously described (Brown et al., Nat Med 7:864-868, 2001).

Neovasculature in de novo adipose tissue was analyzed by acquiring images using mutiphoton laser-scanning microscopy with contrast enhancement of blood vessels by i.v. injection of tetramethylrhodamime labeled dextran (MW 2,000,000). A vertical stack of images were acquired over a depth of 300 μm starting from top layers, which show vessels in de novo adipose tissue, and continued to bottom layers, which show existing blood vessels in underlining host subcutaneous tissue.

Angiogenesis and Adipogenesis in Mouse Model

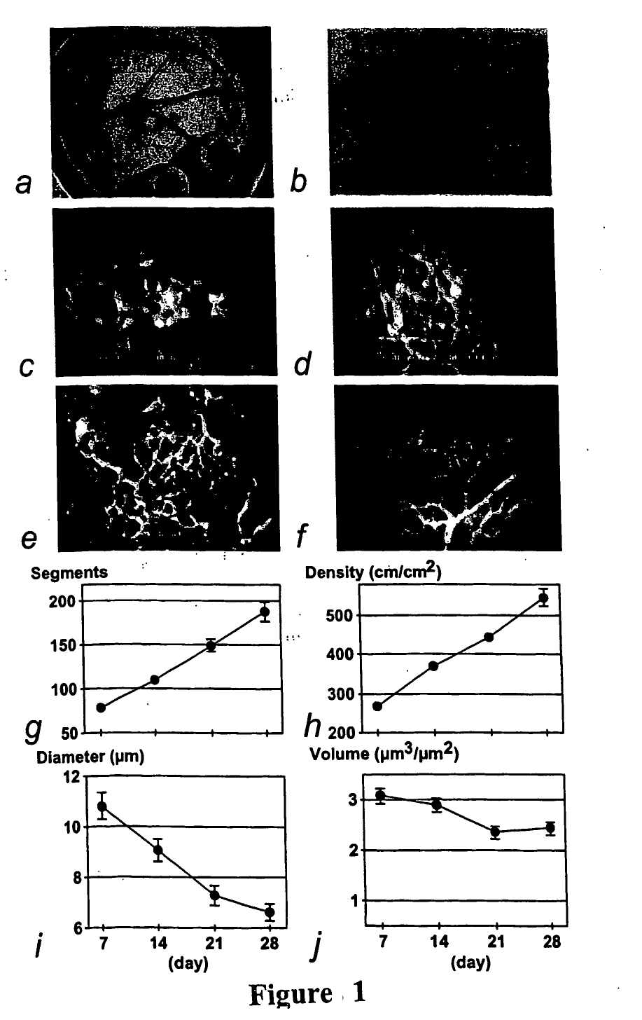

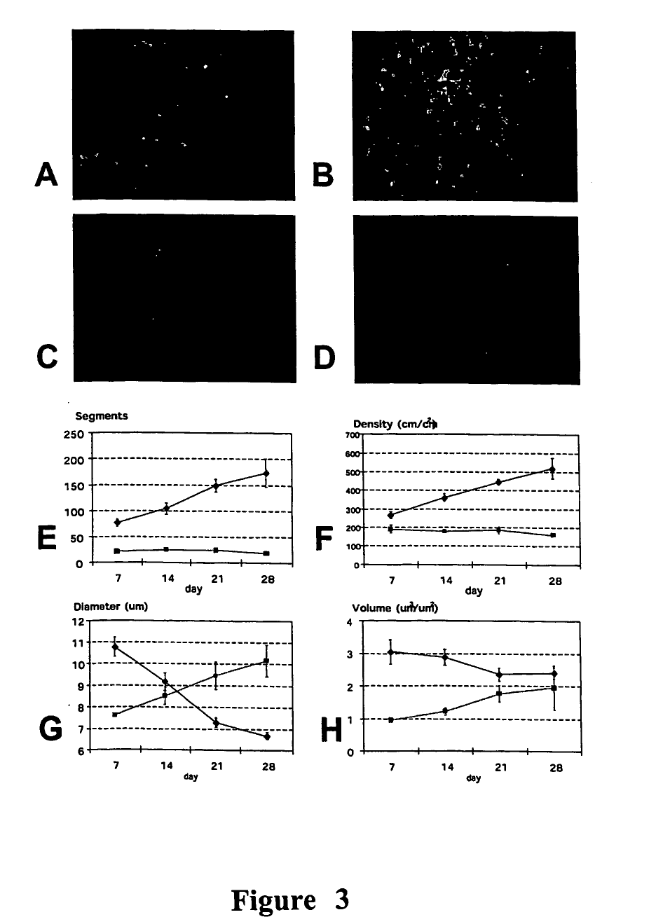

In mice with transplanted preadipocytes, the tissue reddened at the active site of angiogenesis (FIGS. 1A and 1B). Angiogenic vessels appeared in the implanted 3T3-F442 cell pellet on top of the host subcutaneous tissue and striated muscle layer in which pre-existing host vessels were located (FIGS. 12A and 12B). The angiogenic response was specific to the 3T3-F442A preadipocytes, as the parental cell line (NIH 3T3 fibroblasts) did not induce appreciable vessel formation (FIGS. 3A-3H).

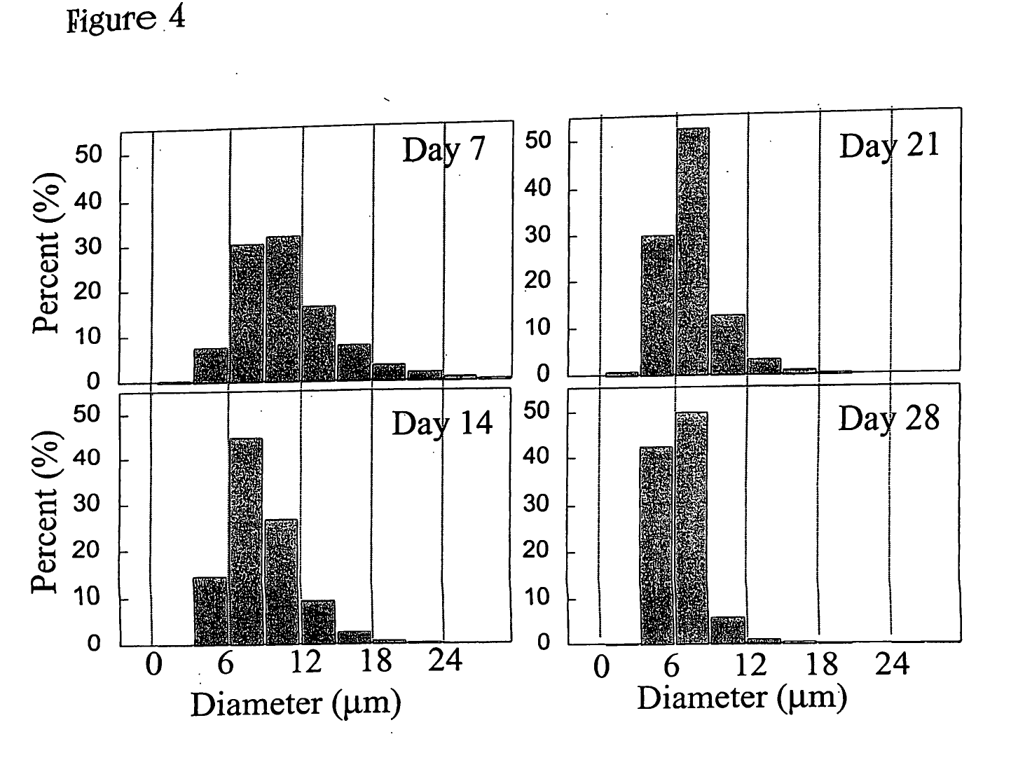

New vessels prompted by the preadipocyte implant appeared to be immature, resembling the vascular plexus during development, with relatively large diameter and no morphological vessel differentiation (FIG. 1C). With time, the vessel network induced by the preadipocyte implant gradually reorganized (FIGS. 1C-1F). Mesh-like patterns of angiogenic vessels turned into a dense capillary network (FIGS. 1C-1E), with arterioles and venules becoming evident (FIG. 1F). The number of blood vessel segments (FIG. 1G) and total length of blood vessels per unit tissue area (FIG. 1H) increased, accompanied by a decrease in mean vessel diameter (FIG. 1I) as the blood vessels remodeled. The total volume of blood vessels per unit tissue area did not change during the remodeling process (FIG. 1J). The blood vessel size distribution narrowed with the remodeling of the vessel network (FIG. 4). These remodeled blood vessels were covered by α-SMA positive cells. The angiogenic response was specific to preadipocytes, as NIH 3T3 fibroblasts did not induce appreciable vessel formation or tissue mass. These findings indicate that preadipocytes induce angiogenesis in vivo and that these angiogenic vessels remodel into an efficient network with mature vessel architecture characterized by small diffusion distance from vessels to parenchymal cells.

One prominent characteristic of cell differentiation is the accumulation of triglyceride-containing vesicles in the cell cytosol. In tissue culture, intra-cellular fat droplets were observed by light microscope (FIG. 13A) and chemically stained by Oil Red O (FIG. 13B). As early as after one week, some of the subcutaneously implanted 3T3-442A cells acquired a typical adipocyte morphology. However, host subcutaneous fat interfered with the Oil Red O staining method in the preadipocyte implantation model.

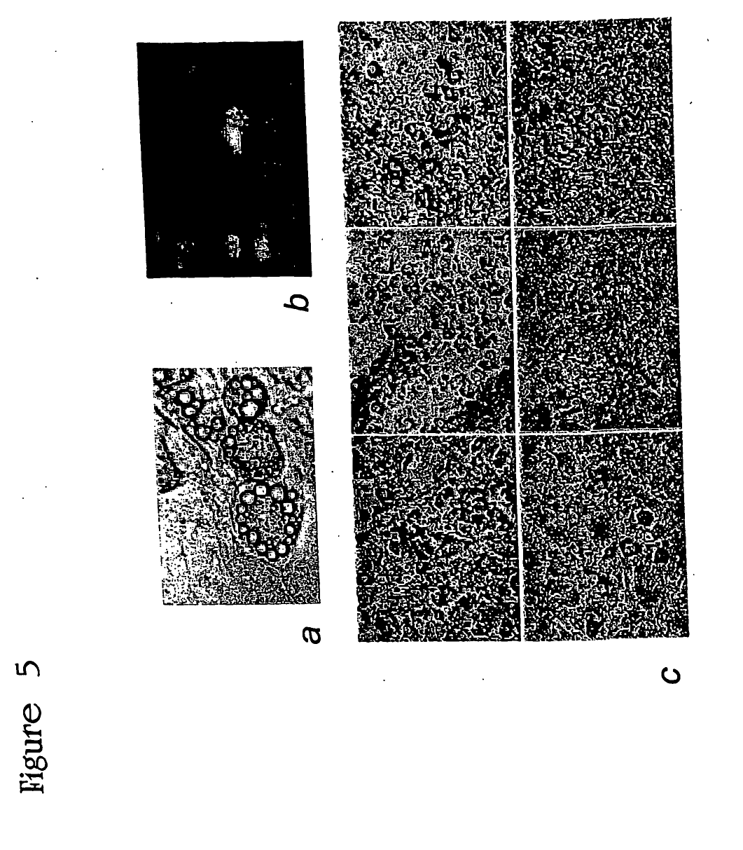

Differentiation into adipocytes was accompanied by accumulation of triglyceride-containing vesicles in the cell cytosol (FIG. 5A). To clearly distinguish between lipid accumulation in implanted cells and host-derived cells in vivo, we labeled 3T3-F442A cells by constitutively expressing the green fluorescent protein (GFP) gene under the control of the EF1α promoter. Cytoplasmic GFP fluorescence allowed us to track implanted cells in vivo and identify the differentiated cells which exhibited a granular fluorescence due to the lipid droplets (FIGS. 5B and 13C). Differentiation into adipocytes began several days after implantation and most of the cells acquired a mature phenotype after four weeks. Differentiation of implanted preadipocytes was further confirmed by expression of the adipocyte-specific aP2 and CD36 (FIG. 10) genes (Bernlohr et al., Biochem. Biophys. Res. Commun. 132:850-855, 1985; Spiegelman et al., J. Biol. Chem. 258:10083-10089, 1983; and Abumrad et al., J. Biol. Chem. 268:17665-17668, 1993). Expression of aP2 in GFP/3T3-442A cell-derived tissue increased with time after implantation into the subcutaneous space of mice (FIG. 13D).

Requirement of PPARγ for Adipogenesis

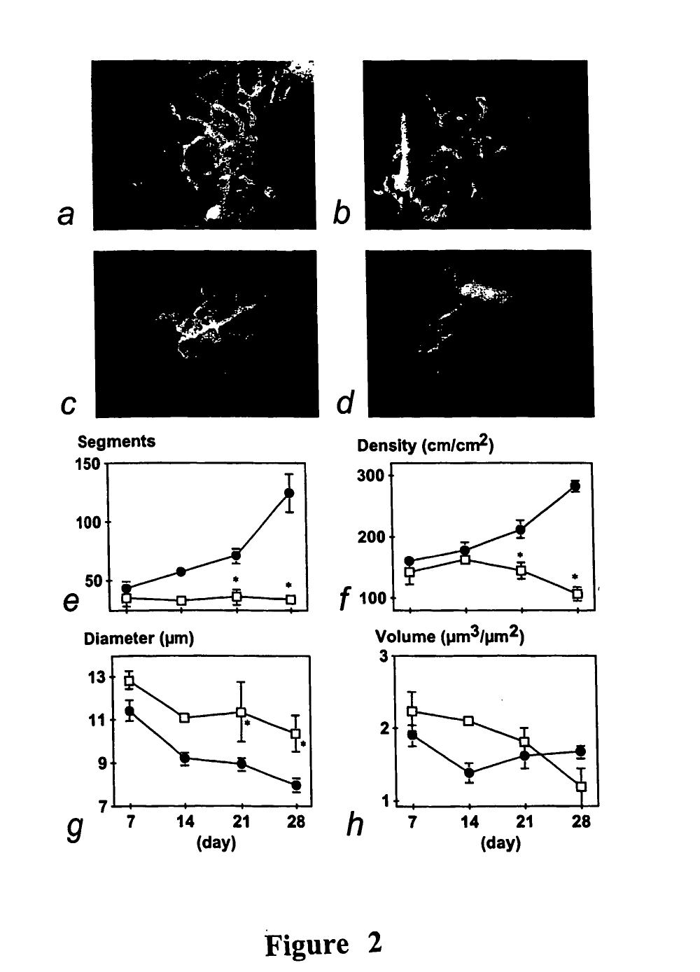

The mechanistic relationship between angiogenesis and adipogenesis has important implications. At the early phase of adipocyte differentiation, expression of lipoprotein lipase (LPL) is increased and preadipocyte factor 1 (pref-1) is decreased, while at least two families of transcriptional factors, CCAAT/enhancer binding protein (C/EBP) and peroxisome proliferator-activated receptor (PPAR), are induced. To elucidate the link between preadipocyte differentiation and angiogenesis, we introduced a dominant negative peroxisome proliferator-activated receptor γ (PPARγ) mutant construct into 3T3-F442A cells prior to implantation using an adenoviral vector, as described previously (Gurnell et al., J. Biol. Chem. 275:5754-5759, 2000). Activation of PPARγ is required for adipocyte differentiation (Lazar, Genes & Dev. 16:1-5, 2002 and Willson et al., Annu. Rev. Biochem. 70:341-367, 2001). Introduction of the mutant receptor, but not mock transfection, prevented the differentiation of 3T3-F442A cells in vitro (FIG. 5C). Mock transfected preadipocytes showed extensive angiogenesis and tissue formation in vivo (FIGS. 2A, 2B, 2E-2H, and 13E), but there was reduced angiogenesis and no fat tissue formation by the implantation of PPARγ dominant negative cells (FIG. 13F). Underlying host blood vessels remained visible for the duration of the experiment (FIGS. 2C and 2D,) when PPARγ dominant negative cells were implanted. These dominant negative cells remained undifferentiated and expressed lower messenger RNA levels of aP2. The tissue size, number of cells, and individual cell size were significantly larger in mock transfected cells compared to those in the PPARγ dominant negative cells (FIG. 13G and 13H). While the vessel segments and vessel density in mock transfected cell implants increased with time, they remained unchanged in the PPARγ-DN cell implanted window (FIGS. 2A-2F). At later time points, mean vessel diameter was significantly larger in the PPARγ-DN group, indicating a lack of vessel remodeling (FIG. 2G). The suppression of PPARγ thus prevents angiogenesis and subsequent vessel remodeling in vivo, rendering the preadipocytes unable to form tissue. Thus, the activation of PPARγ is an indispensable step for adipogenesis and subsequent angiogenesis in vivo.

Expression of VEGF and Other Angiogenesis Related Factors in Adipose Tissue

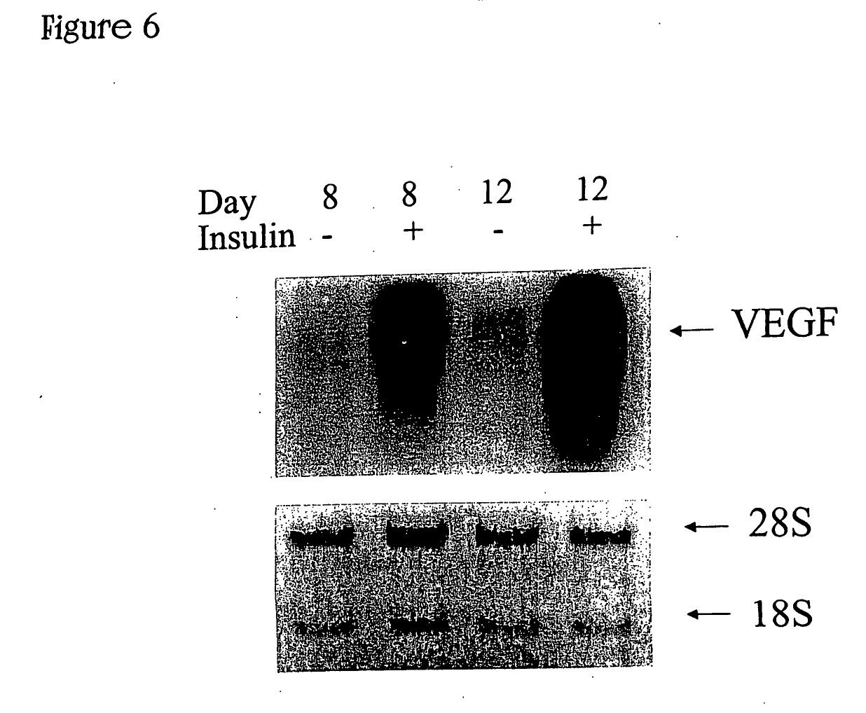

Angiogenesis often precedes adipose tissue formation in developing tissue. VEGF is the most potent and critical angiogenic factor in both physiological and pathological angiogenesis. VEGF is highly expressed in adipose tissue and its expression increases during PPARγ ligand and other stimuli-induced differentiation of preadipocytes into adipocytes (FIGS. 6 and 10) (Zhang et al., J. Surg. Res. 67:147-154, 1997; Claffey et al., J. Biol. Chem. 267:16317-16322, 1992; Soukas et al., J. Biol. Chem. 276:34167-34174, 2001; and Emoto et al., Diabetes 50:1166-1170; 2001). Terminally differentiated mature adipocytes express multiple genes and proteins including aP2 (an adipocyte-specific fatty acid binding protein, originally identified as 422), FAT/CD36, perilipin, adipsin, stearoyl-CoA desaturase (SCD1), glucose transporter (GLUT4), phosphenolpyruvate carboxykinase (PEPCK), and leptin (Gregoire et al., Physiol Rev 78:783-809, 1998; Hwang et al., Annu Rev Cell Dev Biol 13:231-259, 1997; and Rosen et al., AnnuRev Cell Dev Biol 16:145-171, 2000). Among these, the adipocyte specific aP2 gene is a downstream target of PPARγ activation and is the most widely used adipocyte differentiation marker (Bernlohr et al., Biochem Biophys Res Commun 132:850-855, 1985 and Spiegelman et al., J Biol Chem 258:10083-10089, 1983). In agreement with these data, we found expression of VEGF and various other angiogenesis-related genes (FIG. 10) in 3T3-F442A cell-derived tissue in vivo. VEGF-A expression increased during in vitro differentiation of adipocytes and was higher than expression of the various other pro-angiogenic genes that were measured in 3T3-F442A cell-derived tissue in vivo. The kinetics of aP2 expression was also determined to confirm differentiation of 3T3-F442A preadipocytes.

For this analysis of expression of angiogenesis-related genes in vivo, 1.5×10⁷ cells suspended in 100 μl of PBS were injected in the flank of SCID mice (Mandrup et al., Proc Natl Acad Sci USA 94:4300-4305, 1997). For the anti-adipogenesis studies, mice were divided into three groups with the following cell implants: GFP/3T3-F442A, GFP/3T3-F442A expressing PPARγ DN, and GFP/3T3-F442A mock- transfected. For the anti-angiogenesis experiments, GFP/3T3-F442A cells were implanted in three groups of mice. Fat pad formation was allowed to occur for four weeks, then mice were sacrificed, and the tissue was harvested. The tissue formed by the implanted preadipocytes was recovered using microscissors and fluorescence microscope-guided dissection. Tissue samples were snap-frozen for subsequent RNA extraction.

Total RNA was extracted from cells and the recovered tissue samples using Triazol (Gibco BRL, Grand Islands, N.Y.), following the protocol recommended by the manufacturer. The GFP expression in tissue samples was confirmed by RT-PCR. Primers for RT-PCR and Northern probes are shown in FIG. 9. Ten micrograms of total RNA was separated on a 1% agarose/1× MOPS/2% fomaldehyde gel, transferred to nylon membranes in 10× SSC, and UV cross-linked to the membrane. Northern blots were hybridized with random-primed ³²P-labeled probes in QuickHybr Solution (Stratagene, La Jolla Calif.) at 68° C. for one hour. Hybridized blots were washed twice at high stringency in a solution of 0.1×SSC/0.1% sodium dodecylsulfate (SDS) at 55° C. Autoradiography was performed for 1-2 days using a Kodak X-Omat AR film. FIG. 10 summarizes the relative expression levels of several angiogenesis-related genes in preadipocytes, adipocytes, and PPARγ DN treated cells.

Tissue samples were also harvested and fixed at four weeks after implantation. Frozen sections were used for fluorescence immunostaining using a PE-anti-α-SMA antibody (Sigma) and for constitutive GFP cell tracking. DAPI (Molecular probes) was used as a counterstain for the cell nuclei. Quantitation of the GFP-tissue area was performed in five non-sequential transverse sections of the fat tissue generated in the skin chambers by calculating the area of tissue in the perimeter of GFP-positive tissue (3 spots/section, n=5 mice). The area of nuclei was considered representative of the cell number within this tissue. Images were captured using a ×20 objective, an Olympus microscope, a CCD camera, and the OpenLab software. After processing images with Adobe Photoshop software, binary images were quantitated for tissue/cell area using a macro designed by Dr. L. L. Munn in NIH Image software. Resin embedding and routine toluidine-blue counterstaining was used for morphological analyses. The results of this analysis are shown in FIGS. 13E-13G, which demonstrate that PPARγ dominant negative leads to a decrease in cell number compared to control tissues.

Effect of Exogenous VEGF on Adipogenesis In Vitro

Adipose cell differentiation, proliferation, and survival in vitro were not significantly affected by exogenous VEGF (e.g., recombinant mouse VEGF obtained from R&D Systems Minneapolis, Minn.), even at doses as high as 100 ng/ml rmVEGF₁₆₅, added to the culture media (FIGS. 7A, 7B, 8A, and 8B). VEGFR2 expression was undetectable in the preadipocytes. Thus, VEGF signaling may not directly mediate adipogenesis; although, for example, neuropilin-1 was detected in preadipocytes (FIG. 10). Thus, other molecular and microenvironmental changes associated with angiogenesis and/or secondary to VEGF signaling may potentiate adipogenesis in vivo.

VEGF Signaling Mediates Adipocyte Differentiation in a Paracrine Manner

To test the hypothesis that VEGFR1 signaling induces endothelial cell derived factors which potentiate adipose tissue formation, murine endothelial cells (ATCC, Manassa, Va.) were cultured as recommended by the provider for 24 hours by adding recombinant murine VEGF. The media was changed every other day. Cells were harvested at day 11, when cell differentiation started to become apparent, to measure aP2 expression levels. The conditioned media from endothelial cells cultured in the presence of VEGF accelerated the differentiation of preadipocytes, based on an almost three-fold increase in aP2 expression (FIG. 8D).

To assess the paracrine effects of VEGF on cell proliferation, preadipocytes were plated as described for FIG. 8B with conditioned media from endothelial cells cultured in the presence of VEGF and IgG. At day 5, the assay was perform as described, and the optical density for the cells cultured in non-conditioned media was used as a measure of viability. The conditioned media from endothelial cells cultured in the presence of VEGF increased preadipocyte survival/proliferation based on the MTT assay (FIG. 8C). An inhibitor of VEGFR1 signaling can be tested to see if it reverses these effects of VEGF. Such a reversal indicates that VEGFR1 signaling in vascular endothelial cells potentiates preadiopocyte differentiation in a paracrine fashion and explain, in part, the mechanism by which decreasing VEGFR1 activity induces inhibition of adipogenesis in vivo.

Interplay Between Adipose Tissue Formation, Angiogenesis, and Vessel Remodeling

Our data illustrate the complex interplay between adipose tissue formation, angiogenesis, and vessel remodeling. Angiogenesis is needed for efficient preadipocyte differentiation, but angiogenesis is not triggered without PPARγ activation and subsequent adipocyte differentiation. To elucidate the cyclic feedback mechanisms we performed gene array analysis on these tissues at various time points (FIG. 10). Ang-1 was not detectable in mature adipose tissue (FIG. 10). Although we could not detect Ang-1 in our model, 3T3-L1, another preadipocyte cell line, showed increased Ang-1 expression accompanying adipogenesis (Stacker et al., Growth Factors 18:177-191, 2000). Ang-2 was expressed in both preadipocytes and adipocytes in vitro and significantly upregulated by PPARγ-dominant negative transfection (FIG. 10). Adipogenesis may also be mediated by (I) auto- and paracrine effects of other angiogenic growth factor signaling in preadipocytes or (ii) interactions between the matrix associated with angiogenic vessels and preadipocytes (FIG. 10; Varzaneh et al., Metabolism 43:906-912, 1994 and Lilla et al., Am. J. Pathol. 160:1551-1554, 2002).

Research in the last decade or so has uncovered pro- and anti-angiogenic growth factors expressed by adipose tissues or adipocytes, including VEGF-A, VEGF-B, VEGF-C, angiopoietin (Ang)-1, Ang-2, PAI-1, TGFβ, leptin, and maspin. Expression and activity of these factors are also interrelated. For example, leptin induces expression of Ang-2 and stimulates VEGF-induced angiogenesis but represses VEGF-B expression. Temporal and spatial balances of these factors are important for angiogenesis and subsequent vessel remodeling during adipose tissue development. Ang-2 destabilizes existing vessels and induces angiogenesis in the presence of VEGF and other angiogenic factors. On the other hand, activation of the Tie2 receptor by Ang-1 maturates and stabilizes blood vessels and thus, mediates vessel remodeling. Expression of angiopoietins in adipose tissue may depend on the cell differentiation state, site of growth, and external stimuli.

Because PPARγ is crucial for adipocyte differentiation, PPARγ-DN was used to inhibit differentiation of implanted 3T3-F442A preadipocytes. Blocking the PPARγ pathway in preadipocytes inhibited not only differentiation into adipocytes, but also angiogenesis. It is noted that PPARγ regulates different genes/functions in different cell and tissue types: exogenous PPARγ ligands actually inhibits angiogenesis. PPARγ ligands alter VEGF-A expression positively in adipocytes and vascular smooth muscle cells, but negatively in certain tumor cells. The VEGF promoter has not been shown to possess a peroxisome proliferators response element. Furthermore, PPARγ ligands inhibit growth and/or migration of vascular endothelial cells, smooth muscle cells, monocytes, and certain tumor cells. PPARγ ligands also have been previously shown to reduce VEGFR1 and VEGFR2 expression on vascular endothelial cells. Thus, PPARγ ligands inhibit angiogenesis through direct effects on endothelial cells without any change in VEGF level (Murata et al., Arch Opthalmol 119:709-717, 2001).

There is a growing body of evidence showing that blood vessels are more than a carrier of nutrients and passive filters of blood and tissue. Angiogenesis precedes development and repair of organs. Secreting factors from vascular endothelial cells induce proliferation and differentiation of preadipocytes, liver organogenesis, pancreas differentiation, and liver protection. We found that VEGF signaling in vascular endothelial cells induces the secretion of factors that mediate survival/proliferation and differentiation of preadipocytes. Stimulation of mature hepatocytes are predominantly mediated through VEGFR1 signaling in liver sinusoidal endothelial cells (LeCouter et al., Science 299:890-893, 2003). Maintenance of established adipose tissue can be mediated through different signaling pathways in the vascular endothelial cells.

Interactions between extracellular matrix associated with angiogenic vessels and preadipocytes may mediate adipogenesis as well. When Matrigel supplemented with bFGF is implanted, angiogenesis is induced, followed by adipose precursor cells recruitment and fat pad formation (Kawaguchi et al., Proc Natl Acad Sci USA 95:1062-1066, 1998). Microvascular endothelial cells have been previously shown to secrete extracellular matrix components that promote preadipocyte differentiation. Remodeling of extracellular matrix organization is important for both angiogenesis and adipogenesis. Expression of metalloproteases such as MMP 2 and MMP 9 are increased during adipocyte differentiation, and both endogenous and exogenous metalloproteases induce adipogenesis. On the other hand, tissue inhibitor of metalloproteinases-3 (TIMP-3) deficient mice exhibit increased adipose reconstitution during mammary involution. Previously, TIMP-3 has been shown to inhibit angiogenesis by blockage of VEGF binding to VEGFR2. Inhibition of adipogenesis by TIMP-3 could be through a paracrine mechanism of endothelial VEGFR2 signaling.

Finally, an increased supply of nutrients from newly formed vessels is required for advancement of adipocyte differentiation and tissue formation. Furthermore, the HIF-1-regulated gene DEC1/Stra13 inhibits PPARγ gene expression under hypoxia. Tissue oxygenation by angiogenesis and/or vessel remodeling may accelerate adipogenesis by increasing HIF-1 degradation and subsequently potentiating PPARγ activation.

A salient observation emerging from this study comes from the remodeling and maturation of angiogenic vessels. Unlike the aberrant angiogenesis driven by excess and/or unbalanced angiogenic factors that occurs in pathologic conditions such as tumor angiogenesis (Carmeliet et al., supra), the new blood vessels mature into a normal network during adipose tissue formation in this model. This is remarkable since “normal” vasculature is rarely generated in currently available tissue engineering models. Furthermore, our results suggest that the molecular and metabolic microenvironment associated with functional, mature blood vessels potentiates preadipocyte differentiation and adipose tissue formation. This reconfirms that generation of normal microcirculatory units is indispensable for organogesis. The new adipogenesis-organogenesis model described herein is ideal to address the mechanisms of normalization and maturation of blood vessels, and to develop and test novel strategies for tissue engineering, organogenesis, and therapeutic angiogenesis. As described below, interfering with this process allows identification of compounds useful to treat or prevent obesity.

Inhibition of Angiogenesis and/or Adipogenesis by Candidate Compounds Such as Anti-VEGFR1 Antibodies

Based on the above results, it is reasonable to conclude that angiogenesis and vessel remodeling mediated by VEGF-VEGFR1 signaling potentiate adipocyte differentiation. Thus, compounds (e.g., anti-VEGFR1 antibodies) that decrease VEGFR1 activity or block the binding of VEGF to VEGFR1 inhibit adipogenesis and thus prevent or reduce excess body fat. The effect of an anti-VEGFR1 antibody or any other VEGFR1 inhibitor on angiogenesis, adipogenesis, or expression of angiogenesis or adipogenesis associated factors (e.g., aP2) can be tested using the standard assays described herein.

For VEGFR1 inhibition studies, dorsal chamber bearing mice are implanted with preadipocytes as described above and divided into two groups. In one group, rat anti-mouse VEGFR1 antibody administration (MF-1, ImClone Systems Inc., New York, N.Y., 5-50 bodyweight, i.p.) is started on the day of implantation and preferably continued every three days for 30 days. In the control group, the same dose of non-specific isotype-matched rat IgG is given on a similar schedule. Other groups of candidate compounds (e.g., small molecules) and control compounds (or vehicle controls) may be tested similarly.

To measure the ability of a candidate compound to inhibit angiogenesis, the number of non-branching blood vessel segments, functional vascular density, vessel diameter, and vessel volume density can be measured in the presence and absence of the candidate compound as described above. Additionally, the inhibition of mRNA or protein expression of angiogenesis related factors can be measuring using standard methods, such as the gene chip array described herein or western blotting with antibodies reactive with angiogenesis related proteins (see, for example, Ausubel et al., supra). An anti-VEGFR1 antibody or any other VEGFR1 inhibitor is expected to significantly inhibit both angiogenesis and subsequent vessel remodeling in GFP/3T3-F442A preadipocyte implants. In contrast, robust angiogenesis and vessel maturation is expected in control treated animals.

Candidate compounds can also be tested for their ability to inhibit adipogenesis using any of several standard methods. For example, the inhibition of the accumulation of a fat droplet in preadipocyte can be measuring using in vivo microscopy, and the inhibition of lipid formation can be measured by staining with Oil Red 0 red as described herein. Additionally, the ability of a candidate compound to decrease the expression of factors that (I) promote differentiation of preadipocytes into adipocytes, (ii) are required this differentiation, or (iii) are specific for adipocytes can be measured using standard methods (see, for example, Gregoire et al., Physiological Reviews 78:783-809, 1998). For example, the expression of early genes (e.g., PPARγ and C/EBP) or late phase genes (e.g., perilipin, aP2, and FAT/CD36) can be measured. PPARγ and C/EBP are transcription factors that promote adipogenesis; PPARγ, but not C/EBP, is required for adipogenesis. The late phase gene perilipin is specific for adipocytes. As an alternative to measuring the endogenous mRNA or protein expression of these genes, reporter constructs that contain a promoter of one of genes linked to a reporter gene can be used to measure changes in expression of the gene. For example, preadipocytes can be genetically modified by the introduction of a vector contain the leptin or aP2 promoter linked to a lacZ or CAT reporter gene (Cheneval et al., Proc. Natl. Acad. Sci. USA 88:8465-8469, 1991; Hollenberg et al., J. of Biol. Chem. 272:5283-5290, 1997). Immunohistological analysis can be used to measure the expression of the reporter gene in the presence and absence of a candidate compound. A promoter (e.g., the leptin or aP2 promoter) can also be linked to the coding region of green fluorescent protein (Cheneval et al., supra; Hollenberg et al., supra) so that promoter activity can be monitored in vivo without having to harvest tissues from the mouse. Preferred candidate compounds inhibit adipogenesis based on the results of one or more of the above assays.

Other Assays and Animal Models for Testing Compounds of the Invention

As described above, one or more candidate compounds can be tested for their effect on angiogenesis, adipogenesis, or obesity using the mouse model described herein. Alternatively, various genetically engineered obese mice can be used to determine the effect of compounds on obesity. Exemplary mice models of obesity include mice with a heterozygous or homozygous mutation in one or more of the following genes: obese (ob), diabetes (db), tubby (tub), fat, or Agouti, (see, for example, North, Current Opinion in Genetics & Development 9:283-288, 1999). A compound or a combination of compounds can also be tested in standard human clinical trials.

The efficacy of a compound in reducing excess body fat in animal or primate models or in humans can be monitored using standard methods. For example, the body mass index can be used to monitor a subject's weight. The amount of excess body fat can also be approximated by measuring subcutaneous fat (e.g., by measuring the thickness of a skin fold). If desired, a CAT scan or MRI can be used to more accurately measure the amount of body fat. Serum leptin levels should be proportional to the amount of body fat; thus, leptin levels can also be measured to monitor changes in body fat over time.

Administration of Therapies

A therapy of the invention may be administered to humans, domestic pets, livestock, or other animals with a pharmaceutically acceptable diluent, carrier, or excipient, in unit dosage form.

The compounds optionally may be administered as pharmaceutically acceptable salts, such as non-toxic acid addition salts or metal complexes that are commonly used in the pharmaceutical industry. Examples of acid addition salts include organic acids such as acetic, lactic, pamoic, maleic, citric, malic, ascorbic, succinic, benzoic, palmitic, suberic, salicylic, tartaric, methanesulfonic, toluenesulfonic, or trifluoroacetic acids or the like; polymeric acids such as tannic acid, carboxymethyl cellulose, or the like; and inorganic acid such as hydrochloric acid, hydrobromic acid, sulfuric acid phosphoric acid, or the like. Metal complexes include zinc, iron, and the like.

The chemical compounds for use in such therapies may be produced and isolated as described herein or by any standard technique known to those in the field of medicinal chemistry. Conventional pharmaceutical practice may be employed to provide suitable formulations or compositions to administer the identified compound to patients suffering from a higher than desired total body weight or a higher than desired percentage of body fat. Administration may begin before or after the patient is symptomatic.

Any appropriate route of administration may be employed. For example, the therapy may be administered either directly to fat tissue (for example, by injection) or systemically (for example, by any conventional administration technique). Preferably, the therapy is administered using a controlled-release microchip, microparticle extended-release formulation, polymeric nanoparticle, or transdermal delivery system (as described, for example, in LaVan et al., Nature Reviews 1:77-84, 2000 or Santini et al., Nature 397:335-338, 1999). Administration of the compounds may also be oral, topical parenteral, intravenous, intraarterial, subcutaneous, intramuscular, intracranial, intraorbital, ophthalmalic, intraventricular, intracapsular, intraspinal, intracisternal, intraperitoneal, or intranasal. Alternatively, the compounds may be administered as part of a suppository. Therapeutic formulations may be in the form of liquid solutions or suspensions; for oral administration, formulations may be in the form of tablets or capsules; and for intranasal formulations, in the form of powders, nasal drops, or aerosols. The compounds in a combination therapy may be administered simultaneously or sequentially. For example, one or more compounds in a combination therapy can be administered until the compound(s) normalize the blood vessel network of fat tissue and thereby increase the accessibility of the fat tissue to other therapeutic agents, and then one or more additional compounds can be administered instead of, or in addition to, the originally administered compound(s). The dosage of the therapeutic compounds in a pharmaceutically acceptable formulation depends on a number of factors, including the size and health of the individual patient. The dosage to deliver may be determined by one skilled in the art. For example, compounds that are administered as part of a combination therapy of the invention are typically administered at a dose equal to or at least 25, 50, or 75% lower than the corresponding dose for the compound when it is used individually. An exemplary dosing regimen for an anti-VEGFR1 antibody (e.g., MF-1, ImClone) is a dose of 1-100 mg/kg or 5-50 mg/kg every 3 days or 1-20 mg/kg every day. Preferably, the antibody is injected into the mammal.

Methods well known in the art for making formulations are found, for example, in “Remington: The Science and Practice of Pharmacy” ((19th ed.) ed. A. R. Gennaro AR., 1995, Mack Publishing Company, Easton, Pa.). Formulations for parenteral administration may, for example, contain excipients, sterile water, saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

If desired, treatment with a compound identified according to the methods described above, may be combined with more traditional therapies for decreasing total body weight or percentage of body fat (e.g., diet, exercise, or appetite suppressant).

### Other Embodiments

From the foregoing description, it will be apparent that variations and modifications may be made to the invention described herein to adopt it to various usages and conditions. Such embodiments are also within the scope of the following claims.

All publications, patent applications, and patents mentioned in this specification are herein incorporated by reference to the same extent as if each independent publication, patent, or patent application was specifically and individually indicated to be incorporated by reference.

What is claimed is:

1. A screening method for determining whether a compound is useful for treating, stabilizing, or preventing a condition in a mammal selected from the group consisting of a higher than desired total body weight and a higher than desired percentage of body fat, said method comprising measuring VEGFR1 activity in a cell, tissue, or mammal in the presence and absence of said compound, whereby said compound is determined to treat, stabilize, or prevent said condition if said compound decreases VEGFR1 activity.

2-20. (canceled)

21. The method of claim 1, wherein said compound is a member of a library of at least 5 compounds, all of which are simultaneously administered to said cell, tissue, or mammal.

22. The method of claim 1, wherein said compound decreases the level of VEGFR1 mRNA or protein, an activity of VEGFR1, the half-life of VEGFR1 mRNA or protein, or the binding of VEGFR1 to a receptor or to another molecule.

23. The method of claim 1, wherein said compound is an anti-VEGFR1 antibody.

24. The method of claim 1, further comprising administering said compound to a mammal in need of said treatment.

25. The method of claim 24, wherein said mammal is obese.

26. The method of claim 24, wherein the percentage of body fat in said mammal decreases by at least 10%.

27. The method of claim 24, wherein said mammal is a human.

28. A method of treating, stabilizing, or preventing a higher than desired total body weight or a higher than desired percentage of body fat in a mammal, said method comprising administering to said mammal a compound that decreases VEGFR1 activity in an amount sufficient to treat, reduce, or prevent a higher than desired total body weight or a higher than desired percentage of body fat.

29. The method of claim 28, wherein said compound decreases the level of VEGFR1 mRNA or protein, an activity of VEGFR1, the half-life of VEGFR1 mRNA or protein, or the binding of VEGFR1 to a receptor or to another molecule.

30. The method of claim 28, wherein said compound is an anti-VEGFR1 antibody.

31. The method of claim 28, wherein said compound inhibits angiogenesis in said mammal.

32. The method of claim 28, wherein said compound inhibits differentiation of preadipocytes in said mammal.

33. The method of claim 28, wherein at least two compounds that decrease VEGFR1 activity are administered to said mammal.

34. The method of claim 28, wherein said compound is administered intravenously, parenterally, subcutaneously, intramuscularly, ophthalmicly, intraventricularly, intraperitoneally, orally, topically, or intranasally to said mammal.

35. The method of claim 28, further comprising administering a compound that inhibits angiogenesis to said mammal.

36. The method of claim 28, further comprising administering a compound that inhibits VEGF signaling to said mammal.

37. The method of claim 28, wherein said mammal is obese.

38. The method of claim 28, wherein the percentage of body fat in said mammal decreases by at least 10%.

39. The method of claim 28, wherein said mammal is a human.
